(19) United States
(12) Patent Application Publication
(10) Pub. No.: US 2017/0190753 A1
(43) Pub. Date: Jul. 6, 2017

(54) RECOMBINANT PROTEINS HAVING FACTOR H ACTIVITY

(71) Applicant: LABORATOIRE FRANCAIS DU FRACTIONNEMENT ET DES BIOTECHNOLOGIES, Les Ulis (FR)

(72) Inventor: Toufik ABACHE, Santes (FR)

(21) Appl. No.: 15/105,829

(22) PCT Filed: Dec. 19, 2014
(86) PCT No.: PCT/FR2014/053484

§ 371 Date: Sep. 26, 2016

(30) Foreign Application Priority Data
Dec. 20, 2013 (FR) 1363351

(51) Int. Cl. C07K 14/47; A01K 67/027

(52) U.S. Cl. CPC C07K 14/472; A01K 67/0275; C07K 2319/21; C07K 2319/02

(57) ABSTRACT

The invention relates to a recombinant protein having factor H activity.

The invention relates to a recombinant protein having factor H activity.

### BACKGROUND OF THE INVENTION

Factor H is a 155 kDa plasma protein the main function of which is regulation of alternative complement pathway activity. Factor H consists of 20 short consensus repeat (SCR) domains (also called CCP or SHUSHI domains) of about 60 amino acids linked together by a short linker sequence of 3 to 8 amino acids. SCR domains 1-4 (SCR1-4) have the activity of accelerating the dissociation of C3 and C5 convertases and the activity of regulating factor I, which permits inactivation of the C3b protein. These N-terminal domains are sufficient to regulate C3 convertase activity in the fluid phase, but SCR19-20 are necessary to factor H activity on the cell surface. Other factor H SCRs can also contribute more or less directly to factor H activity, some containing binding sites for other molecules such as heparan sulfates and glycosaminoglycans, pentraxins (CRP, PTX3), fibromodulin or malondialdehyde. Some of these domains also contain glycosylation sites, which can contribute to the molecule's half-life and the efficacy of its production in recombinant form, others may have an important role for the three-dimensional conformation of factor H, such as for example SCR12, SCR13, SCR14, which confer on factor H a specific hairpin-shaped folding structure (Schmidt et al., J Mol Biol. 2010 Jan. 08; 395(1): 105-122).

Factor H is a promising therapeutic factor for the treatment of numerous diseases associated with dense C3 deposits or with complement activation or uncontrolled inflammation. However, in certain indications such as age-related macular degeneration (ARMD), membranoproliferative glomerulonephritis (MPGN) type II, atypical hemolytic uremic syndrome (aHUS) or certain autoimmune diseases, the use of factor H may be limited because of certain disadvantages: bioavailability, pharmacokinetics and pharmacodynamics, immunogenicity, binding to heparan sulfates, binding to unidentified ligands, binding to pathogens that enable them to evade the immune system (S. pneumoniae, N. meningitidis, etc.) and factor H self-association. These disadvantages are related to the molecular properties of factor H and thus to the organization of the SCR domains of factor H.

A factor H derivative combining SCR1 to SCR4 with SCR19 and SCR20 of factor H (Hebecker et al., Journal of Immunology 2013, Jun. 10, published online) has been proposed. In this factor H derivative constructed to direct factor H activity on the cell surface, the two domains of complement regulation and of surface recognition are linked together by the 6 natural amino acids found between the fourth cysteine of SCR4 and the first cysteine of SCR19.

However, other factor H domains may also be necessary in order to obtain a therapeutic efficacy superior to that of factor H, for example in order to obtain better binding to cells, to conserve or increase the half-life of FH or to conserve or promote the characteristic hairpin-shaped folding of FH. There is thus a need for molecules derived from factor H, said molecules derived from factor H having factor H activity, having an advantage over factor H or not having the disadvantages of factor H, and whose activity would preferably be conserved or improved compared to that of factor H.

### SUMMARY OF THE INVENTION

The applicant responds here to that need by proposing recombinant proteins derived from factor H having a rearrangement of the number and the organization of the SCR domains of factor H which conserve factor H activity. The recombinant proteins according to the invention comprise at least SCR1-4 (SCR1, SCR2, SCR3 and SCR4, in that order), SCR19 and SCR20 of factor H.

An object of the invention thus relates to a recombinant protein having factor H activity, comprising, from the N-terminus to the C-terminus, a first amino acid sequence comprising at least SCR1 to SCR4 of factor H, and a second amino acid sequence comprising at least SCR19 and SCR20 of factor H, said recombinant protein not being a natural factor H, and provided that if the first sequence consists of SCR1 to SCR4 and the second sequence consists of SCR19 and SCR20, the linker is a synthetic linker.

The invention also has as an object a nucleic acid encoding a recombinant protein according to the invention, a vector comprising such a nucleic acid, a host cell comprising such a nucleic acid or such a vector and a transgenic animal the genome of which comprises such a nucleic acid.

The invention also has as an object a recombinant protein according to the invention, for treating diseases due to uncontrolled inflammation or uncontrolled C3 convertase deposition. More generally, the recombinant protein according to the invention may be useful for treating any disease for which anti-complement activity of factor H is beneficial.

### DETAILED DESCRIPTION OF THE INVENTION

In an embodiment, the present invention relates to a recombinant protein derived from factor H having a rearrangement of SCR domains, in terms of both their number and their organization, said recombinant protein comprising, in this order, a first amino acid sequence comprising SCR1-4 of factor H and a second amino acid sequence comprising SCR19 and SCR20 of factor H. The first and/or second amino acid sequence can further comprise one or more other rearranged SCR domains of factor H.

In an embodiment, one or more SCR domains of factor H can be present in several copies. For example, a recombinant protein according to the invention can include one, two, three, or more than three copies of the same SCR, for example one, two, three or more than three copies of SCR1, SCR2, SCR3, SCR4, SCR5, SCR6, SCR7, SCR8, SCR9, SCR10, SCR11, SCR12, SCR13, SCR14, SCR15, SCR16, SCR17, SCR18, SCR19 and SCR20 of factor H. According to a particular embodiment, the recombinant protein according to the invention comprises one, two, three or more than three copies of SCR7 of factor H, in particular of SCR7 of the Y402 variant of factor H. Another variant includes a recombinant protein comprising multiple copies of a set of SCR domains. For example, the recombinant protein according to the invention can comprise one, two, three or more than three repeats of SCR1 to SCR4, i.e., the protein has the motif (SCR1-SCR4)ₙ, n being an integer equal to 1, 2, 3 or greater than 3, in particular n being equal to 1, 2 or 3. In another embodiment, the domain or the set of domains present in several copies is not a repeat of the domains or the sets of domains, but can be present in the recombinant protein separated by other SCR domains. In other words, the invention relates in particular to recombinant proteins comprising, in this order, SCR1-SCR4, SCR7, SCR7 and SCR19-SCR20, or SCR1-SCR4, SCR7, SCR1-SCR4, and SCR19-20, or any other possible combination of these domains.

The factor H from which the protein according to the invention is derived can be any factor H the activity of which is that of natural factor H. In particular, by “factor H” is meant here any protein having the amino acid sequence of native human factor H or that from another species (for example bovine, porcine, canine, murine). Preferably, the recombinant protein is derived from human factor H. The term also includes any recombinant, derivative or mutant having a sequence substantially homologous to native factor H. The expression “sequence substantially homologous to” comprises any sequence subject to one or more substitutions, additions and/or deletions, preferably conservative. The expression “conservative substitutions, additions and/or deletions” refers to any replacement, addition or removal of an amino acid residue with another, with no major alteration of the general conformation and/or the biological activity of factor H. Conservative substitution comprises, but is not limited to, replacement with an amino acid having similar properties (such as, for example, shape, polarity, hydrogen bonding potential, acidity, basicity, hydrophobicity, etc.). Amino acids having similar properties are well-known in the art. The term “factor H” further includes the natural allelic variations and/or the isoforms of factor H found naturally in individuals of the same species, and any form or degree of glycosylation or other post-translational modification. Also included in the term “factor H” are homologues or derivatives of factor H that have the same activity, or superior biological activity compared to the activity of the wild form and/or that have sequence identity of at least 80%, preferably at least 85%, more preferably of at least 90%, more preferably at least 95%, more preferably at least 98%, more preferably at least 99%.

The factor H variant used to design the recombinant protein of the present invention can in particular be a variant mentioned in the Internet site http://www.uniprot.org/uniprot/P08603.

The anti-complement activity of factor H translates to regulation of the alternative complement pathway by maintaining a basal level of C3b molecules. Factor H competes with factor B for binding to C3b and accelerates the dissociation of the alternative C3 convertase (C3bBb) already formed. It acts as a factor I cofactor in the proteolysis of C3b, free or bound to the cell surface, which leads to the inactive form C3bi. Thus, immune complexes consisting of an antigen-antibody complex associated with complement component C3b or with factors activating the alternative complement pathway (bacterial surfaces, infected cells, yeasts, parasites, lipopolysaccharides, endotoxins) can no longer activate the subsequent complement cascade (components C5-C9). The term “biological activity” of factor H thus includes here the ability to inhibit C3 convertase and/or to serve as factor I cofactor, resulting in the inhibition of complement cascade activation.

In a particular embodiment, the recombinant protein conserves the biological activity of human plasma factor H.

The biological activity of human plasma factor H comprises the regulation of factor I activity, the inhibition of the formation of alternative C3 convertase and the acceleration of the dissociation of C3 convertase.

The methods for determining the above biological activities are known to the person skilled in the art.

In a more particular embodiment, said recombinant protein conserves the biological activity of human plasma factor H for accelerating the dissociation of C3 convertase. The amount of C3 convertase dissociated can be determined as a function of the concentration of recombinant protein added. IC₅₀ is determined from the equation calculated for a sigmoid of variable slope.

In another more particular embodiment, said recombinant protein conserves the biological activity of plasma factor H for controlling factor I activity.

Furthermore, the biological activity of factor H can be evaluated by measuring the activity of protection of red blood cells from lysis by the complement according to procedures well-known in the state of the art.

The sequence SEQ ID NO: 1 represents the amino acid sequence of the Y402 variant of human factor H in which the amino acid at position 402 is a tyrosine. This sequence does not comprise a signal peptide. The sequence SEQ ID NO: 2 represents the amino acid sequence of the H402 variant of human factor H in which the amino acid at position 402 is a histidine. This sequence does not comprise a signal peptide. In an embodiment, one or more SCR domains of the recombinant protein according to the invention are derived from either of these two variants. According to a particular embodiment, the recombinant protein comprises SCR7 of the Y402 variant of factor H. According to variants of this embodiment, the first sequence of the recombinant protein comprises SCR1 to SCR7, SCR1 to SCR8 or SCR1 to SCR9 of factor H, SCR7 in this first sequence being SCR7 of the Y402 variant of factor H. In these variant embodiments, other SCR domains can be derived from the Y402 variant of factor H or from another variant of factor H. Furthermore, the first sequence can in particular be combined with a second amino acid sequence comprising SCR19 and SCR20, SCR18 to SCR20, SCR17 to SCR20 or SCR16 to SCR20 of factor H, the second amino acid sequence being in particular derived from the Y402 variant or the H402 variant of factor H. These recombinant proteins include the Y402 polymorphism present in SCR7.

In an embodiment, the recombinant protein according to the invention comprises, in this order, SCR1, SCR2, SCR3, SCR4, SCR19 and SCR20 of factor H. In this embodiment, SCR4 and SCR19 are linked together by a non-natural (or synthetic) linker consisting of a sequence comprising between 2 and 20 amino acids. By “non-natural (or synthetic) linker” is meant in particular a linker that does not correspond to the sequence found between the fourth cysteine of SCR4 and the first cysteine of SCR19. In particular, the amino acid sequence of the linker is selected in such a way that it is encoded by a nucleic acid that, when it is introduced into a cloning and/or expression vector, comprises a unique restriction site (see below). For example, the linker can have the sequence GASG (SEQ ID NO: 3).

According to an embodiment, the first or the second amino acid sequence of the recombinant protein according to the invention comprises one or more additional SCR domains of factor H selected from SCR5, SCR6, SCR7, SCR8, SCR9, SCR10, SCR11, SCR12, SCR13, SCR14, SCR15, SCR16, SCR17 and SCR18. The order of these domains in the recombinant protein can correspond to the order of the same domains in natural factor H. Alternatively, the order of the domains can be different from that of natural factor H.

The amino acid sequence of SCR1 (amino acids 21 to 80) of the Y402 variant of human factor H is represented by the sequence SEQ ID NO: 4 (CNELPPRRNTEILTGSWSDQTYPEGTQAIYKCRPGYRSLGNVIMVCRKGEWVALNPLRKC). The sequence of the SCR1 introduced into the protein according to the invention has at least 85%, in particular at least 90%, in particular at least 95%, in particular at least 99% identity to the sequence SEQ ID NO: 4. In an embodiment, the sequence of the SCR1 introduced into the protein according to the invention is that represented in the sequence SEQ ID NO: 4.

The amino acid sequence of SCR2 (amino acids 85 to 141) of the Y402 variant of human factor H is represented by the sequence SEQ ID NO: 5 (CGHPGDTPFGTFTLTGGNVFEYGVKAVYTCNEGYQLLGEINYRECDTDGWTNDIPIC). The sequence of the SCR2 introduced into the protein according to the invention has at least 85%, in particular at least 90%, in particular at least 95%, in particular at least 99% identity to the sequence SEQ ID NO: 5. In an embodiment, the sequence of the SCR2 introduced into the protein according to the invention is that represented in the sequence SEQ ID NO: 5.

The amino acid sequence of SCR3 (amino acids 146 to 205) of the Y402 variant of human factor H is represented by the sequence SEQ ID NO: 6 (CLPVTAPENGKIVSSAMEPDREYHFGQAVRFVCNSGYKIEGDEEMHCSDDGFWSKEKPKC). The sequence of the SCR3 introduced into the protein according to the invention has at least 85%, in particular at least 90%, in particular at least 95%, in particular at least 99% identity to the sequence SEQ ID NO: 6. In an embodiment, the sequence of the SCR3 introduced into the protein according to the invention is that represented in the sequence SEQ ID NO: 6.

The amino acid sequence of SCR4 (amino acids 210 to 262) of the Y402 variant of human factor H is represented by the sequence SEQ ID NO: 7 (CKSPDVINGSPISQKIIYKENERFQYKCNMGYEYSERGDAVCTESGWRPLPSC). The sequence of the SCR4 introduced into the protein according to the invention has at least 85%, in particular at least 90%, in particular at least 95%, in particular at least 99% identity to the sequence SEQ ID NO: 7. In an embodiment, the sequence of the SCR4 introduced into the protein according to the invention is that represented in the sequence SEQ ID NO: 7.

The amino acid sequence of SCR5 (amino acids 266 to 320) of the Y402 variant of human factor H is represented by the sequence SEQ ID NO: 8 (CDNPYIPNGDYSPLRIKHRTGDEITYQCRNGFYPATRGNTAKCTSTGWIPAPRC). The sequence of the SCR5 introduced into the protein according to the invention has at least 85%, in particular at least 90%, in particular at least 95%, in particular at least 99% identity to the sequence SEQ ID NO: 8. In an embodiment, the sequence of the SCR5 introduced into the protein according to the invention is that represented in the sequence SEQ ID NO: 8.

The amino acid sequence of SCR6 (amino acids 325 to 385) of the Y402 variant of human factor H is represented by the sequence SEQ ID NO: 9 (CDYPDIKHGGLYHENMRRPYFPVAVGKYYSYYCDEHFETPSGSYWDHIHCTQDGWSPAVPC). The sequence of the SCR6 introduced into the protein according to the invention has at least 85%, in particular at least 90%, in particular at least 95%, in particular at least 99% identity to the sequence SEQ ID NO: 9. In an embodiment, the sequence of the SCR6 introduced into the protein according to the invention is that represented in the sequence SEQ ID NO: 9.

The amino acid sequence of SCR7 (amino acids 389 to 442) of the Y402 variant of human factor H is represented by the sequence SEQ ID NO: 10 (CYFPYLENGYNQNYGRKFVQGKSIDVACHPGYALPKAQTTVTCMENGWSPTPRC).

The sequence of the SCR7 introduced into the protein according to the invention has at least 85%, in particular at least 90%, in particular at least 95%, in particular at least 99% identity to the sequence SEQ ID NO: 10. In an embodiment, the sequence of the SCR7 introduced into the protein according to the invention is that represented in the sequence SEQ ID NO: 10.

The amino acid sequence of SCR8 (amino acids 448 to 505) of the Y402 variant of human factor H is represented by the sequence SEQ ID NO: 11 (CSKSSIDIENGFISESQYTYALKEKAKYQCKLGYVTADGETSGSITCGKDGWSAQPTC). The sequence of the SCR8 introduced into the protein according to the invention has at least 85%, in particular at least 90%, in particular at least 95%, in particular at least 99% identity to the sequence SEQ ID NO: 11. In an embodiment, the sequence of the SCR8 introduced into the protein according to the invention is that represented in the sequence SEQ ID NO: 11.

The amino acid sequence of SCR9 (amino acids 509 to 564) of the Y402 variant of human factor H is represented by the sequence SEQ ID NO: 12 (CDIPVFMNARTKNDFTWFKLNDTLDYECHDGYESNTGSTTGSIVCGYNGWSDLPIC). The sequence of the SCR9 introduced into the protein according to the invention has at least 85%, in particular at least 90%, in particular at least 95%, in particular at least 99% identity to the sequence SEQ ID NO: 12. In an embodiment, the sequence of the SCR9 introduced into the protein according to the invention is that represented in the sequence SEQ ID NO: 12.

The amino acid sequence of SCR10 (amino acids 569 to 623) of the Y402 variant of human factor H is represented by the sequence SEQ ID NO: 13 (CELPKIDVHLVPDRKKDQYKVGEVLKFSCKPGFTIVGPNSVQCYHFGLSPDLPIC).

The sequence of the SCR10 introduced into the protein according to the invention has at least 85%, in particular at least 90%, in particular at least 95%, in particular at least 99% identity to the sequence SEQ ID NO: 13. In an embodiment, the sequence of the SCR10 introduced into the protein according to the invention is that represented in the sequence SEQ ID NO: 13.

The amino acid sequence of SCR11 (amino acids 630 to 674) of the Y402 variant of human factor H is represented by the sequence SEQ ID NO: 14 (CGPPPELLNGNVKEKTKEEYGHSEVVEYYCNPRFLMKGPNKIQCVDGEWTTLPVC).

The sequence of the SCR11 introduced into the protein according to the invention has at least 85%, in particular at least 90%, in particular at least 95%, in particular at least 99% identity to the sequence SEQ ID NO: 14. In an embodiment, the sequence of the SCR11 introduced into the protein according to the invention is that represented in the sequence SEQ ID NO: 14.

The amino acid sequence of SCR12 (amino acids 691 to 744) of the Y402 variant of human factor H is represented by the sequence SEQ ID NO: 15 (CGDIPELEHGWAQLSSPPYYYGDSVEFNCSESFTMIGHRSITCIHGVWTQLPQC). The sequence of the SCR12 introduced into the protein according to the invention has at least 85%, in particular at least 90%, in particular at least 95%, in particular at least 99% identity to the sequence SEQ ID NO: 15. In an embodiment, the sequence of the SCR12 introduced into the protein according to the invention is that represented in the sequence SEQ ID NO: 15.

The amino acid sequence of SCR13 (amino acids 753 to 803) of the Y402 variant of human factor H is represented by the sequence SEQ ID NO: 16 (CKSSNLIILEEHLKNKKEFDHNSNIRYRCRGKEGWIHTVCINGRWDPEVNC). The sequence of the SCR13 introduced into the protein according to the invention has at least 85%, in particular at least 90%, in particular at least 95%, in particular at least 99% identity to the sequence SEQ ID NO: 16. In an embodiment, the sequence of the SCR13 introduced into the protein according to the invention is that represented in the sequence SEQ ID NO: 16.

The amino acid sequence of SCR14 (amino acids 811 to 864) of the Y402 variant of human factor H is represented by the sequence SEQ ID NO: 17 (CPPPPQIPNSHNMTTTLNYRDGEKVSVLCQENYLIQEGEEITCKDGRWQSIPLC). The sequence of the SCR14 introduced into the protein according to the invention has at least 85%, in particular at least 90%, in particular at least 95%, in particular at least 99% identity to the sequence SEQ ID NO: 17. In an embodiment, the sequence of the SCR14 introduced into the protein according to the invention is that represented in the sequence SEQ ID NO: 17.

The amino acid sequence of SCR15 (amino acids 869 to 926) of the Y402 variant of human factor H is represented by the sequence SEQ ID NO: 18 (CSQPPQIEHGTINSSRSSQESYAHGTKLSYTCEGGFRISEENETTCYMGKWSSPPQC). The sequence of the SCR15 introduced into the protein according to the invention has at least 85%, in particular at least 90%, in particular at least 95%, in particular at least 99% identity to the sequence SEQ ID NO: 18. In an embodiment, the sequence of the SCR15 introduced into the protein according to the invention is that represented in the sequence SEQ ID NO: 18.

The amino acid sequence of SCR16 (amino acids 931 to 984) of the Y402 variant of human factor H is represented by the sequence SEQ ID NO: 19 (CKSPPEISHGVVAHMSDSYQYGEEVTYKCFEGFGIDGPAIAKCLGEKWSHPPSC). The sequence of the SCR16 introduced into the protein according to the invention has at least 85%, in particular at least 90%, in particular at least 95%, in particular at least 99% identity to the sequence SEQ ID NO: 19. In an embodiment, the sequence of the SCR16 introduced into the protein according to the invention is that represented in the sequence SEQ ID NO: 19.

The amino acid sequence of SCR17 (amino acids 989 to 1043) of the Y402 variant of human factor H is represented by the sequence SEQ ID NO: 20 (CLSLPSFENAIPMGEKKDVYKAGEQVTYTCATYYKMDGASNVTCINSRWTGRPTC). The sequence of the SCR17 introduced into the protein according to the invention has at least 85%, in particular at least 90%, in particular at least 95%, in particular at least 99% identity to the sequence SEQ ID NO: 20. In an embodiment, the sequence of the SCR17 introduced into the protein according to the invention is that represented in the sequence SEQ ID NO: 20.

The amino acid sequence of SCR18 (amino acids 1048 to 1102) of the Y402 variant of human factor H is represented by the sequence SEQ ID NO: 21 (CVNPPTVQNAYIVSRQMSKYPSGERVRYQCRSPYEMFGDEEVMCLNGNWTEPPQC). The sequence of the SCR18 introduced into the protein according to the invention has at least 85%, in particular at least 90%, in particular at least 95%, in particular at least 99% identity to the sequence SEQ ID NO: 21. In an embodiment, the sequence of the SCR18 introduced into the protein according to the invention is that represented in the sequence SEQ ID NO: 21.

The amino acid sequence of SCR19 (amino acids 1109 to 1163) of the Y402 variant of human factor H is represented by the sequence SEQ ID NO: 22 (CGPPPPIDNGDITSFPLSVYAPASSVEYQCQNLYQLEGNKRITCRNGQWSEPPKC).

The sequence of the SCR19 introduced into the protein according to the invention has at least 85%, in particular at least 90%, in particular at least 95%, in particular at least 99% identity to the sequence SEQ ID NO: 22. In an embodiment, the sequence of the SCR18 introduced into the protein according to the invention is that represented in the sequence SEQ ID NO: 22.

The amino acid sequence of SCR20 (amino acids 1167 to 1231) of the Y402 variant of human factor H is represented by the sequence SEQ ID NO: 23 (CVISREIMENYNIALRWTAKQKLYSRTGESVEFVCKRGYRLSSRSHTLRTTCWDGKLEYPTCAKR). The sequence of the SCR18 introduced into the protein according to the invention has at least 85%, in particular at least 90%, in particular at least 95%, in particular at least 99% identity to the sequence SEQ ID NO: 23. In an embodiment, the sequence of the SCR20 introduced into the protein according to the invention is that represented in the sequence SEQ ID NO: 23.

In an embodiment, the first or the second amino acid sequence comprises at least SCR12, SCR13 and SCR14 of factor H. In another embodiment, not one of SCR12, SCR13 and SCR14 is present in the recombinant protein according to the invention. A variant embodiment of the present invention relates to a recombinant protein the SCR domains of which consist, in this order, SCR1, SCR2, SCR3, SCR4, SCR12, SCR13, SCR14, SCR19 and SCR20. Such a protein is represented in the sequence SEQ ID NO: 142.

Each SCR domain included in the first or the second amino acid sequence can be linked to the contiguous SCR or SCRs by means of the natural linker found between said SCR domains, if need be. Alternatively, the linker between each SCR domain of the recombinant protein can be a non-natural linker. The non-natural linker can consist of a sequence comprising between 2 and 20 amino acids, in particular between 3 and 8 amino acids.

Furthermore, the link between the first and the second amino acid sequence can be produced by means of a natural or synthetic linker present at the C-terminal position of the first amino acid sequence or the N-terminal position of the second amino acid sequence. For example, the first and the second polypeptide can be linked together by a linker having the sequence GASG (SEQ ID NO: 3). Furthermore, the first and the second amino acid sequences can be linked together by a linker combining the natural linker sequence following the last domain of the first sequence (i.e., the domain at the C-terminal position of the first sequence) and a synthetic linker such as the linker GASG. The natural linkers found at the C-terminal position of each of the SCR domains of factor H are for example: QKRP (SEQ ID NO: 143) for SCR1; EVVK (SEQ ID NO: 144) for SCR2; VEIS (SEQ ID NO: 145) for SCR3; EEKS (SEQ ID NO: 146) for SCR4; TLKP (SEQ ID NO: 147) for SCR5; LRK for SCR6; IRVKT (SEQ ID NO: 148) for SCR7; IKS for SCR8; YERE (SEQ ID NO: 149) for SCR9; KEQVQS (SEQ ID NO: 150) for SCR10; IVEEST (SEQ ID NO: 151) for SCR11; VAIDKLKK (SEQ ID NO: 152) for SCR12; SMAQIQL (SEQ ID NO: 153) for SCR13; VEKIP (SEQ ID NO: 154) for SCR14; EGLP (SEQ ID NO: 155) for SCR15; IKTD (SEQ ID NO: 156) for SCR16; RDTS (SEQ ID NO: 157) for SCR17; KDSTGK (SEQ ID NO: 158) for SCR18; LHP for SCR19.

According to an embodiment, the first amino acid sequence comprises SCR1, SCR2, SCR3 and SCR4 of factor H.

According to an embodiment, the first amino acid sequence comprises SCR1, SCR2, SCR3, SCR4 and SCR5 of factor H.

According to another embodiment, the first amino acid sequence comprises SCR1, SCR2, SCR3, SCR4, SCR5 and SCR6 of factor H.

According to another embodiment, the first amino acid sequence comprises SCR1, SCR2, SCR3, SCR4, SCR5, SCR6 and SCR7 of factor H.

According to another embodiment, the first amino acid sequence comprises SCR1, SCR2, SCR3, SCR4, SCR5, SCR6, SCR7 and SCR8 of factor H.

According to another embodiment, the first amino acid sequence comprises SCR1, SCR2, SCR3, SCR4, SCR5, SCR6, SCR7, SCR8 and SCR9 of factor H.

According to another embodiment, the first amino acid sequence comprises SCR1, SCR2, SCR3, SCR4, SCR5, SCR6, SCR7, SCR8, SCR9 and SCR10 of factor H.

According to another embodiment, the first amino acid sequence comprises SCR1, SCR2, SCR3, SCR4, SCR5, SCR6, SCR7, SCR8, SCR9, SCR10 and SCR11 of factor H.

According to another embodiment, the first amino acid sequence comprises SCR1, SCR2, SCR3, SCR4, SCR5, SCR6, SCR7, SCR8, SCR9, SCR10, SCR11 and SCR12 of factor H.

According to another embodiment, the first amino acid sequence comprises SCR1, SCR2, SCR3, SCR4, SCR5, SCR6, SCR7, SCR8, SCR9, SCR10, SCR11, SCR12 and SCR13 of factor H.

According to an embodiment, the second amino acid sequence comprises SCR19 and SCR20 of factor H.

According to an embodiment, the second amino acid sequence comprises SCR18, SCR19 and SCR20 of factor H.

According to an embodiment, the second amino acid sequence comprises SCR17, SCR18, SCR19 and SCR20 of factor H.

According to an embodiment, the second amino acid sequence comprises SCR16, SCR17, SCR18, SCR19 and SCR20 of factor H.

According to an embodiment, the second amino acid sequence comprises SCR15, SCR16, SCR17, SCR18, SCR19 and SCR20 of factor H.

According to an embodiment, the second amino acid sequence comprises SCR14, SCR15, SCR16, SCR17, SCR18, SCR19 and SCR20 of factor H.

According to an embodiment, the second amino acid sequence comprises SCR13, SCR14, SCR15, SCR16, SCR17, SCR18, SCR19 and SCR20 of factor H.

According to an embodiment, the second amino acid sequence comprises SCR12, SCR13, SCR14, SCR15, SCR16, SCR17, SCR18, SCR19 and SCR20 of factor H.

According to an embodiment, the second amino acid sequence comprises SCR11, SCR12, SCR13, SCR14, SCR15, SCR16, SCR17, SCR18, SCR19 and SCR20 of factor H.

According to an embodiment, the second amino acid sequence comprises SCR10, SCR11, SCR12, SCR13, SCR14, SCR15, SCR16, SCR17, SCR18, SCR19 and SCR20 of factor H.

According to an embodiment, the second amino acid sequence comprises SCR9, SCR10, SCR11, SCR12, SCR13, SCR14, SCR15, SCR16, SCR17, SCR18, SCR19 and SCR20 of factor H.

According to an embodiment, the second amino acid sequence comprises SCR8, SCR9, SCR10, SCR11, SCR12, SCR13, SCR14, SCR15, SCR16, SCR17, SCR18, SCR19 and SCR20 of factor H.

According to another embodiment, the recombinant protein having factor H activity comprises the first and second amino acid sequences listed in table 1 below, these sequences being separated by a linker, in particular an artificial linker, in particular the linker GASG (SEQ ID NO: 3).

TABLE 1

| First sequence | Second sequence |
|---|---|
| SCR1 to SCR4 | SCR19 to SCR20 |
| SCR1 to SCR4 | SCR18 to SCR20 |
| SCR1 to SCR4 | SCR17 to SCR20 |
| SCR1 to SCR4 | SCR16 to SCR20 |
| SCR1 to SCR4 | SCR15 to SCR20 |
| SCR1 to SCR4 | SCR14 to SCR20 |
| SCR1 to SCR4 | SCR13 to SCR20 |
| SCR1 to SCR4 | SCR12 to SCR20 |
| SCR1 to SCR4 | SCR11 to SCR20 |
| SCR1 to SCR4 | SCR10 to SCR20 |
| SCR1 to SCR4 | SCR9 to SCR20 |
| SCR1 to SCR4 | SCR8 to SCR20 |
| SCR1 to SCR7 | SCR19 to SCR20 |
| SCR1 to SCR7 | SCR18 to SCR20 |
| SCR1 to SCR7 | SCR17 to SCR20 |
| SCR1 to SCR7 | SCR16 to SCR20 |
| SCR1 to SCR7 | SCR15 to SCR20 |
| SCR1 to SCR7 | SCR14 to SCR20 |
| SCR1 to SCR7 | SCR13 to SCR20 |
| SCR1 to SCR7 | SCR12 to SCR20 |
| SCR1 to SCR7 | SCR11 to SCR20 |
| SCR1 to SCR7 | SCR10 to SCR20 |
| SCR1 to SCR7 | SCR9 to SCR20 |
| SCR1 to SCR7 | SCR8 to SCR20 |
| SCR1 to SCR8 | SCR19 to SCR20 |
| SCR1 to SCR8 | SCR18 to SCR20 |
| SCR1 to SCR8 | SCR17 to SCR20 |
| SCR1 to SCR8 | SCR16 to SCR20 |
| SCR1 to SCR8 | SCR15 to SCR20 |
| SCR1 to SCR8 | SCR14 to SCR20 |
| SCR1 to SCR8 | SCR13 to SCR20 |
| SCR1 to SCR8 | SCR12 to SCR20 |
| SCR1 to SCR8 | SCR11 to SCR20 |
| SCR1 to SCR8 | SCR10 to SCR20 |
| SCR1 to SCR8 | SCR9 to SCR20 |
| SCR1 to SCR9 | SCR19 to SCR20 |
| SCR1 to SCR9 | SCR18 to SCR20 |
| SCR1 to SCR9 | SCR17 to SCR20 |
| SCR1 to SCR9 | SCR16 to SCR20 |
| SCR1 to SCR9 | SCR15 to SCR20 |
| SCR1 to SCR9 | SCR14 to SCR20 |
| SCR1 to SCR9 | SCR13 to SCR20 |
| SCR1 to SCR9 | SCR12 to SCR20 |
| SCR1 to SCR9 | SCR11 to SCR20 |
| SCR1 to SCR9 | SCR10 to SCR20 |
| SCR1 to SCR10 | SCR19 to SCR20 |
| SCR1 to SCR10 | SCR18 to SCR20 |
| SCR1 to SCR10 | SCR17 to SCR20 |
| SCR1 to SCR10 | SCR16 to SCR20 |
| SCR1 to SCR10 | SCR15 to SCR20 |
| SCR1 to SCR10 | SCR14 to SCR20 |
| SCR1 to SCR10 | SCR13 to SCR20 |
| SCR1 to SCR10 | SCR12 to SCR20 |
| SCR1 to SCR10 | SCR11 to SCR20 |
| SCR1 to SCR11 | SCR19 to SCR20 |
| SCR1 to SCR11 | SCR18 to SCR20 |
| SCR1 to SCR11 | SCR17 to SCR20 |
| SCR1 to SCR11 | SCR16 to SCR20 |
| SCR1 to SCR11 | SCR15 to SCR20 |
| SCR1 to SCR11 | SCR14 to SCR20 |
| SCR1 to SCR11 | SCR13 to SCR20 |
| SCR1 to SCR11 | SCR12 to SCR20 |
| SCR1 to SCR12 | SCR19 to SCR20 |
| SCR1 to SCR12 | SCR18 to SCR20 |
| SCR1 to SCR12 | SCR17 to SCR20 |
| SCR1 to SCR12 | SCR16 to SCR20 |
| SCR1 to SCR12 | SCR15 to SCR20 |
| SCR1 to SCR12 | SCR14 to SCR20 |
| SCR1 to SCR12 | SCR13 to SCR20 |
| SCR1 to SCR13 | SCR19 to SCR20 |
| SCR1 to SCR13 | SCR18 to SCR20 |
| SCR1 to SCR13 | SCR17 to SCR20 |
| SCR1 to SCR13 | SCR16 to SCR20 |
| SCR1 to SCR13 | SCR15 to SCR20 |
| SCR1 to SCR13 | SCR14 to SCR20 |

According to a particular embodiment, the recombinant protein having factor H activity comprises the first and second amino acid sequences listed in table 2 below, these sequences being separated by a linker, in particular an artificial linker, in particular the linker GASG (SEQ ID NO: 3).

TABLE 2

| First sequence | Second sequence |
|---|---|
| SCR1 to SCR4 | SCR19 to SCR20 |
| SCR1 to SCR4 | SCR16 to SCR20 |
| SCR1 to SCR4 | SCR15 to SCR20 |
| SCR1 to SCR4 | SCR14 to SCR20 |
| SCR1 to SCR4 | SCR11 to SCR20 |
| SCR1 to SCR4 | SCR10 to SCR20 |
| SCR1 to SCR4 | SCR9 to SCR20 |
| SCR1 to SCR4 | SCR8 to SCR20 |
| SCR1 to SCR7 | SCR16 to SCR20 |
| SCR1 to SCR7 | SCR15 to SCR20 |
| SCR1 to SCR7 | SCR14 to SCR20 |
| SCR1 to SCR7 | SCR11 to SCR20 |
| SCR1 to SCR7 | SCR10 to SCR20 |
| SCR1 to SCR7 | SCR9 to SCR20 |
| SCR1 to SCR7 | SCR8 to SCR20 |
| SCR1 to SCR8 | SCR16 to SCR20 |
| SCR1 to SCR8 | SCR15 to SCR20 |
| SCR1 to SCR8 | SCR14 to SCR20 |
| SCR1 to SCR8 | SCR13 to SCR20 |
| SCR1 to SCR8 | SCR11 to SCR20 |
| SCR1 to SCR8 | SCR10 to SCR20 |
| SCR1 to SCR8 | SCR9 to SCR20 |
| SCR1 to SCR9 | SCR19 to SCR20 |
| SCR1 to SCR9 | SCR16 to SCR20 |
| SCR1 to SCR9 | SCR14 to SCR20 |
| SCR1 to SCR9 | SCR13 to SCR20 |
| SCR1 to SCR9 | SCR12 to SCR20 |
| SCR1 to SCR9 | SCR11 to SCR20 |
| SCR1 to SCR9 | SCR10 to SCR20 |
| SCR1 to SCR10 | SCR11 to SCR20 |
| SCR1 to SCR13 | SCR14 to SCR20 |

According to a preferred variant of the invention, the first sequence comprises SCR1 to SCR7 of factor H, and the second sequence is selected from the group consisting of:
SCR9 to SCR20,
SCR11 to SCR20; and
SCR14 to SCR20 of factor H.

According to another preferred variant, the first sequence comprises SCR1 to SCR8 and the second sequence comprises SCR10 to SCR20.

According to another preferred variant, the first sequence comprises SCR1 to SCR4 of factor H and the second sequence comprises SCR16 to SCR20 of factor H, a third sequence comprising SCR7 of factor 7 being between the first and the second sequence, and being more particularly separated from the latter by linkers such as those described above.

According to an embodiment, the first amino acid sequence comprises a signal peptide (SP) at the N-terminal position. The signal peptide can be the natural signal peptide of factor H (MRLLAKIICLMLWAICVA—SEQ ID NO: 24), the signal peptide of a protein different from factor H, or a signal peptide described in the application PCT/2001/050544, in particular the peptide MRWSWIFLLLLSITSANA (SEQ ID NO: 25; or also called SP-MB7 hereinafter). The natural signal peptide of a protein different from human factor H can be a signal peptide selected from the signal peptides of all the proteins secreted in eukaryotes and in particular in mammals and more particularly in humans, like those of immunoglobulins, of growth factors like EPO, of hormones like insulin, of enzymes like trypsinogen, of coagulation factors such as prothrombin. The presence of a signal peptide improves secretion of the recombinant protein in the culture medium.

According to an embodiment, the invention relates to one of the peptides of table 1 in which the first amino acid sequence comprises at the N-terminus such a signal peptide, in particular the natural peptide of factor H having the sequence SEQ ID NO: 24 or the signal peptide SP-MB7 having the sequence SEQ ID NO: 25. According to another embodiment, the recombinant protein according to the invention is selected from one of the proteins of table 1, the first and second sequences being separated by a linker, in particular a GASG linker (SEQ ID NO: 3).

According to a particular embodiment, the recombinant protein according to the invention is selected from one of the proteins listed in table 2 below in which the first amino acid sequence comprises a signal peptide having the sequence SEQ ID NO: 25 and the first and second sequences are separated by a linker having the sequence GASG (SEQ ID NO: 3).

TABLE 3

| SEQ | | | |
|---|---|---|---|
| ID NO: | First sequence | Linker | Second sequence |
| 26 | SP-MB7 + SCR1 to SCR4 | GASG | SCR19 to SCR20 |
| 27 | SP-MB7 + SCR1 to SCR4 | GASG | SCR16 to SCR20 |
| 28 | SP-MB7 + SCR1 to SCR4 | GASG | SCR15 to SCR20 |
| 29 | SP-MB7 + SCR1 to SCR4 | GASG | SCR14 to SCR20 |
| 30 | SP-MB7 + SCR1 to SCR4 | GASG | SCR13 to SCR20 |
| 31 | SP-MB7 + SCR1 to SCR4 | GASG | SCR12 to SCR20 |
| 32 | SP-MB7 + SCR1 to SCR4 | GASG | SCR11 to SCR20 |
| 33 | SP-MB7 + SCR1 to SCR4 | GASG | SCR10 to SCR20 |
| 34 | SP-MB7 + SCR1 to SCR4 | GASG | SCR9 to SCR20 |
| 35 | SP-MB7 + SCR1 to SCR4 | GASG | SCR8 to SCR20 |
| 36 | SP-MB7 + SCR1 to SCR7 | GASG | SCR19 to SCR20 |
| 37 | SP-MB7 + SCR1 to SCR7 | GASG | SCR16 to SCR20 |
| 38 | SP-MB7 + SCR1 to SCR7 | GASG | SCR15 to SCR20 |
| 39 | SP-MB7 + SCR1 to SCR7 | GASG | SCR14 to SCR20 |
| 40 | SP-MB7 + SCR1 to SCR7 | GASG | SCR13 to SCR20 |
| 41 | SP-MB7 + SCR1 to SCR7 | GASG | SCR12 to SCR20 |
| 42 | SP-MB7 + SCR1 to SCR7 | GASG | SCR11 to SCR20 |
| 43 | SP-MB7 + SCR1 to SCR7 | GASG | SCR10 to SCR20 |
| 44 | SP-MB7 + SCR1 to SCR7 | GASG | SCR9 to SCR20 |
| 45 | SP-MB7 + SCR1 to SCR7 | GASG | SCR8 to SCR20 |
| 46 | SP-MB7 + SCR1 to SCR8 | GASG | SCR19 to SCR20 |
| 47 | SP-MB7 + SCR1 to SCR8 | GASG | SCR16 to SCR20 |
| 48 | SP-MB7 + SCR1 to SCR8 | GASG | SCR15 to SCR20 |
| 49 | SP-MB7 + SCR1 to SCR8 | GASG | SCR14 to SCR20 |
| 50 | SP-MB7 + SCR1 to SCR8 | GASG | SCR13 to SCR20 |
| 51 | SP-MB7 + SCR1 to SCR8 | GASG | SCR12 to SCR20 |
| 52 | SP-MB7 + SCR1 to SCR8 | GASG | SCR11 to SCR20 |
| 53 | SP-MB7 + SCR1 to SCR8 | GASG | SCR10 to SCR20 |
| 54 | SP-MB7 + SCR1 to SCR8 | GASG | SCR9 to SCR20 |
| 55 | SP-MB7 + SCR1 to SCR9 | GASG | SCR19 to SCR20 |
| 56 | SP-MB7 + SCR1 to SCR9 | GASG | SCR16 to SCR20 |
| 57 | SP-MB7 + SCR1 to SCR9 | GASG | SCR15 to SCR20 |
| 58 | SP-MB7 + SCR1 to SCR9 | GASG | SCR14 to SCR20 |
| 59 | SP-MB7 + SCR1 to SCR9 | GASG | SCR13 to SCR20 |
| 60 | SP-MB7 + SCR1 to SCR9 | GASG | SCR12 to SCR20 |
| 61 | SP-MB7 + SCR1 to SCR9 | GASG | SCR11 to SCR20 |
| 62 | SP-MB7 + SCR1 to SCR9 | GASG | SCR10 to SCR20 |
| 63 | SP-MB7 + SCR1 to SCR10 | GASG | SCR19 to SCR20 |
| 64 | SP-MB7 + SCR1 to SCR10 | GASG | SCR16 to SCR20 |
| 65 | SP-MB7 + SCR1 to SCR10 | GASG | SCR15 to SCR20 |
| 66 | SP-MB7 + SCR1 to SCR10 | GASG | SCR14 to SCR20 |
| 67 | SP-MB7 + SCR1 to SCR10 | GASG | SCR13 to SCR20 |
| 68 | SP-MB7 + SCR1 to SCR10 | GASG | SCR12 to SCR20 |
| 69 | SP-MB7 + SCR1 to SCR10 | GASG | SCR11 to SCR20 |
| 70 | SP-MB7 + SCR1 to SCR11 | GASG | SCR19 to SCR20 |
| 71 | SP-MB7 + SCR1 to SCR11 | GASG | SCR16 to SCR20 |
| 72 | SP-MB7 + SCR1 to SCR11 | GASG | SCR15 to SCR20 |
| 73 | SP-MB7 + SCR1 to SCR11 | GASG | SCR14 to SCR20 |
| 74 | SP-MB7 + SCR1 to SCR11 | GASG | SCR13 to SCR20 |
| 75 | SP-MB7 + SCR1 to SCR11 | GASG | SCR12 to SCR20 |
| 76 | SP-MB7 + SCR1 to SCR12 | GASG | SCR19 to SCR20 |
| 77 | SP-MB7 + SCR1 to SCR12 | GASG | SCR16 to SCR20 |
| 78 | SP-MB7 + SCR1 to SCR12 | GASG | SCR15 to SCR20 |
| 79 | SP-MB7 + SCR1 to SCR12 | GASG | SCR14 to SCR20 |
| 80 | SP-MB7 + SCR1 to SCR12 | GASG | SCR13 to SCR20 |
| 81 | SP-MB7 + SCR1 to SCR13 | GASG | SCR19 to SCR20 |
| 82 | SP-MB7 + SCR1 to SCR13 | GASG | SCR16 to SCR20 |
| 83 | SP-MB7 + SCR1 to SCR13 | GASG | SCR15 to SCR20 |
| 84 | SP-MB7 + SCR1 to SCR13 | GASG | SCR14 to SCR20 |

According to a particular embodiment, the recombinant protein according to the invention contains a first sequence comprising SCR1 to SCR4 of factor H, and a second sequence selected from the group consisting of:
SCR16 to SCR20,
SCR15 to SCR20;
SCR10 to SCR20; and
SCR8 to SCR20 of factor H.

According to another embodiment, the recombinant protein according to the invention contains a first sequence comprising SCR1 to SCR7 of factor H, and a second sequence comprising SCR16 to SCR20 of factor H, said second sequence being preferably selected from the group consisting of:
SCR15 to SCR20;
SCR14 to SCR20;
SCR11 to SCR20;
SCR10 to SCR20;
SCR9 to SCR20; and
SCR8 to SCR20 of factor H.

According to another variant, the invention relates to a recombinant protein as described above, the first sequence comprising SCR1 to SCR8 of factor H, and the second sequence comprising SCR16 to SCR20 of factor H, said second sequence preferably comprising SCR10 to SCR20 of factor H.

According to another embodiment, the recombinant protein according to the invention contains a first sequence comprising SCR1 to SCR4 of factor H and a second sequence comprising SCR16 to SCR20 of factor H, a third sequence comprising SCR7 of factor H being between the first and the second sequence.

According to a variant of the invention, the first sequence comprises a signal peptide having the sequence SEQ ID NO: 25 and SCR1 to SCR7 of factor H, and the second sequence is selected from the group consisting of:
SCR9 to SCR20;
SCR11 to SCR20; and
SCR14 to SCR20 of factor H.

In a particular embodiment of this variant, the first and the second sequences are separated by a linker having the sequence GASG.

According to another preferred variant, the first sequence comprises a signal peptide having the sequence SEQ ID NO: 25 and SCR1 to SCR8 and the second sequence comprises SCR10 to SCR20. In a particular embodiment of this variant, the first and the second sequences are separated by a linker having the sequence GASG.

According to another preferred variant, the first sequence comprises a signal peptide having the sequence SEQ ID NO: 25 and SCR1 to SCR4 of factor H and the second sequence comprises SCR16 to SCR20 of factor H, a third sequence comprising SCR7 of factor H being between the first and the second sequence, and being separated from the latter by linkers such as those described above. In a particular embodiment of this variant, the first and the third sequences, and the third and the second sequences are separated by a linker having the sequence GASG. Such a sequence is represented in SEQ ID NO: 159.

The present invention also relates to a pharmaceutical composition comprising a recombinant protein according to the invention.

The invention also has as an object a nucleic acid construct encoding a recombinant protein having factor H activity as described above.

Advantageously, the nucleic acids encoding the recombinant proteins according to the invention were the subject of codon optimization.

The purpose of codon optimization is to replace the natural codons with codons the transfer RNAs (tRNAs) of which bearing the amino acids are the most frequent in the cell type concerned. Mobilizing frequently encountered tRNAs has the major advantage of increasing the rate of translation of the messenger RNAs (mRNAs) and thus increasing the final titer (Carton J M et al., Protein Expr Purif, 2007). Sequence optimization also affects the prediction of mRNA secondary structures which can slow reading by the ribosomal complex. Sequence optimization also has an impact on G/C percentage, which is directly related to the half-life of the mRNAs and thus to their potential for being translated (Chechetkin, J. of Theoretical Biology 242, 2006 922-934).

Codon optimization can be carried out by substitution of the natural codons using codon usage tables for mammals and more particularly for Homo sapiens. There are algorithms on the Internet made available by suppliers of synthetic genes (DNA2.0, GeneArt, MWG, Genscript) which make it possible to carry out this sequence optimization.

On a purely illustrative basis, a sequence having been the subject of codon optimization is represented by the sequence SEQ ID NO: 85. This sequence comprises the natural signal peptide of factor H.

The nucleic acids according to the invention can comprise a unique restriction site between the two nucleic acid sequences encoding the first and the second amino acid sequence of the recombinant protein according to the invention. This unique restriction site can in particular correspond to the NheI site present in the portion encoding the GASG linker (nucleic sequence: GCCGCTAGCGCC (SEQ ID NO: 86), the underlined portion corresponding to the NheI site which corresponds to the amino acids AS mentioned above. This unique restriction site makes it possible to envisage an improvement of the recombinant proteins produced, in particular by facilitated introduction of one or more additional SCR domains in the protein sequence corresponding to said restriction site, or by introduction of an amino acid sequence different from an SCR domain. It can in particular be another protein domain not belonging to natural factor H, or a linker of different size and sequence (in particular a longer linker).

The nucleic acids encoding the recombinant protein according to the invention can be constructed according to any method known to the person skilled in the art of molecular biology. Advantageously, however, said nucleic acid is constructed in two steps according to a process proper to the present invention. For example, a bank of nucleic acids cloned into cloning or expression vectors can be assembled. Each vector of the bank contains a sequence encoding one of the first or second amino acid sequences making up the recombinant protein according to the invention. Thus is described a vector comprising sequences encoding a first amino acid sequence comprising at least the sequences of SCR1 to SCR4 of factor H (or N-Ter vector). Also described is a vector comprising sequences encoding a second amino acid sequence comprising at least the sequences of SCR19 and SCR20 of factor H (or C-Ter vector).

The N-Ter and C-Ter vectors are designed so as to comprise unique restriction sites useful for the excision and then the assembly of the nucleic acid fragments encoding each part of the recombinant protein in a single vector. Constructs permitting the expression of the proteins in table 2 above can thus be produced from 8 N-Ter vectors and 10 C-Ter vectors. This strategy is described in the examples below.

The nucleic acids according to the invention can also comprise any useful sequence, in particular any sequence permitting optimization of the expression or the secretion of the recombinant protein or for facilitating the cloning and the subcloning of the nucleic acids of the invention. For example, the nucleic acid may comprise at the 5′ end a unique restriction site, a coding sequence and/or a sequence encoding a signal peptide. At the 3′ end, it may in particular be useful to introduce a sequence encoding an amino acid motif useful for the labeling or the purification of the recombinant protein, for example a histidine tag, and one or more restriction sites.

In an embodiment, the nucleic acid construct according to the invention comprises a sequence encoding a signal peptide selected from:
a nucleic acid represented by the sequence SEQ ID NO: 87 and encoding the natural signal peptide of factor H, or
a nucleic acid represented by the sequence SEQ ID NO: 88 or by a sequence having at least 85%, in particular 90%, particularly 95% sequence identity to the sequence SEQ ID NO: 88, and encoding the factor H signal peptide (optimized natural SP), or
a nucleic acid encoding a natural signal peptide of a protein different from factor H, or
a nucleic acid encoding the signal peptide encoded by the sequence SEQ ID NO: 89 (described in the application PCT/FR2011/050544) or by a sequence having at least 85%, in particular 90%, particularly 95% sequence identity to the sequence SEQ ID NO: 89.

The sequence SEQ ID NO: 88 or a sequence having at least 85%, in particular 90%, particularly 95% sequence identity to the sequence SEQ ID NO: 88 is a sequence obtained by codon optimization starting from the sequence SEQ ID NO: 87.

The nucleic acid represented by the sequence SEQ ID NO: 89 or by a sequence having at least 85%, in particular 90%, particularly 95% sequence identity to the sequence SEQ ID NO: 89 encodes the artificial signal peptide SP-MB7 described above.

In an embodiment, the nucleic acid construct encoding the recombinant protein according to the invention comprises, or consists of:
a nucleic acid encoding a signal peptide, in particular a nucleic acid represented by the sequence SEQ ID NO: 87 and encoding the natural signal peptide of factor H, or a nucleic acid represented by the sequence SEQ ID NO: 88 or by a sequence having at least 85%, in particular 90%, particularly 95% sequence identity to the sequence SEQ ID NO: 88, and encoding the factor H signal peptide (optimized natural SP) or a nucleic acid encoding a natural signal peptide of a protein different from factor H, or a nucleic acid encoding the signal peptide encoded by the sequence SEQ ID NO: 89 (described in the application PCT/FR2011/050544) or by a sequence having at least 85%, in particular 90%, particularly 95% sequence identity to the sequence SEQ ID NO: 89;
a nucleic acid encoding at least SCR1, SCR2, SCR3 and SCR4 of factor H;
a nucleic acid encoding a linker, in particular a GASG linker (SEQ ID NO: 3), said nucleic acid encoding a linker comprising a unique restriction site, in particular a NheI site;
a nucleic acid encoding at least SCR19 and SCR20 of factor H.

According to an embodiment, the nucleic acid construct permits the expression of a recombinant protein selected from one of the sequences SEQ ID NO: 26 to SEQ ID NO: 84.

The invention also relates to an expression vector comprising the nucleic acid construct described above, functionally linked to expression control sequences of said nucleic acid. The control sequences can in particular comprise a promoter (in particular a CMV promoter), an enhancer, and any sequence known to the person skilled in the art useful for permitting the expression of the recombinant protein in a eukaryotic cell, in particular a mammalian cell.

To that end, the invention further relates to a recombinant eukaryotic cell, in particular a mammalian cell, more particularly a non-human cell (e.g., CHO) or human cell, transformed by means of the nucleic acid construct or the expression vector according to the invention. In an advantageous embodiment of the present invention, the recombinant protein as described above is produced in the PER.C6® cell line or an HEK cell line, in particular the HEK 293F cell line.

The PER.C6® cell line arises from human primary retina cells in which an adenovirus DNA fragment Ad5 containing both the E1A gene and E1B gene is inserted into the cells by means of a vector. This adenovirus DNA fragment confers immortality on the cells into which it is inserted, via the E1B protein which inhibits the p53 protein. The E1A protein, in turn, has a tropism for the viral promoter hCMV and permits its transactivation and the potentiation of the gene sequence which will be inserted at the 3′ end of the latter and which may be the recombinant protein according to the invention.

The present invention particularly relates to the use of the PER.C6® cell line for implementing a process for preparing a recombinant protein having factor H activity, in particular one of the proteins having the sequence SEQ ID NO: 26 to SEQ ID NO: 84.

The present invention also particularly relates to the use of the HEK 293F cell line for implementing a process for preparing a recombinant protein according to the invention, in particular one of the recombinant proteins represented by one of the sequences SEQ ID NO: 26 to SEQ ID NO: 84.

The invention also relates to a process for producing a recombinant protein having factor H activity. The process according to the invention, in particular one of the proteins represented by the sequences SEQ ID NO: 26 to 84, said process comprising culturing a recombinant cell according to the invention transformed by a vector comprising the nucleic acid construct according to the invention encoding said recombinant protein.

The vector comprising such a nucleic acid can be any expression vector for eukaryotic cell lines known to the person skilled in the art.

The transformation of the cell line can be implemented using electroporation, AMAXA-type nucleofection, a “gene gun” or using a transfection agent known to the person skilled in the art, such as cationic agents, liposomes or polymers such as Fectin or the agent PEI.

In a particular embodiment, the process according to the present invention comprises the following steps:

(i) transfecting a eukaryotic cell with an expression vector comprising a nucleic acid construct encoding the recombinant protein, in order to obtain a transfected cell,

(ii) culturing said transfected cell, in order to obtain the expression of the recombinant protein in the culture medium.

Said expression vector can contain an antibiotic resistance gene in order to allow the selection of transfected cells during the establishment of cells that stably produce the protein of interest.

In an embodiment, the recombinant protein according to the invention is produced at a concentration of 10 mg/L or greater as detected by ELISA of the culture supernatant, after 7 days of production in batch mode.

The purification of the recombinant protein can be implemented by one-, two- or several-step chromatography techniques. One-step purification can be an ion-exchange column or an affinity column (heparin, factor H ligand or anti-factor H antibody). Two-step purification can be a step of cation-exchange column chromatography followed by a step of anion-exchange column chromatography or a step of anion-exchange column chromatography followed by a step of cation-exchange column chromatography or a step of ion-exchange column chromatography followed by a step of affinity column chromatography or a step of affinity column chromatography followed by a step of ion-exchange column chromatography. Purification employing more than two steps can be carried out by a combination of these various chromatographies. A step of diafiltration, ultrafiltration or gel filtration can be carried out in addition. The purity of a product after such a purification can reach 99% purified product.

The proteins according to the invention can also be produced in the milk of non-human transgenic animals, such as goats, rabbits, ewes, cows or pigs. In this case, the secretion of the proteins by the mammary glands, enabling their secretion in the milk of the transgenic mammal, involves controlling the expression of the proteins according to the invention in a tissue-dependent manner. Such methods of control are well-known to the person skilled in the art. The expression is controlled by means of sequences permitting the expression of the protein toward a particular tissue of the animal. They are in particular WAP, beta-casein and beta-lactoglobulin promoter sequences and signal peptide sequences. The process for extracting proteins of interest from the milk of transgenic animals is described in the patent EP 0 264 166.

Another aspect of the invention also relates to the use of a recombinant protein according to the invention as a medicinal product.

The invention also relates to the use of a recombinant protein according to the invention for the manufacture of a medicinal product intended for treating a disease involving undesirable or inappropriate complement activity, in particular for treating diseases due to uncontrolled inflammation or uncontrolled C3b deposition. The invention also relates to a method for treating a disease involving undesirable or inappropriate complement activity, comprising administering a recombinant protein according to the invention to a patient in need of such treatment. The term “treatment” includes both curative treatment and prophylactic treatment of the disease. Curative treatment is defined as treatment leading to a cure or treatment alleviating, improving and/or eliminating, reducing and/or stabilizing the symptoms of a disease or the suffering caused by it. Prophylactic treatment includes both treatment leading to the prevention of a disease and treatment reducing and/or delaying the incidence of a disease or the risk of its occurrence. The invention relates in particular to treating a disease selected from the group consisting of age-related macular degeneration (ARMD), periodontitis, lupus erythematosus, lupus nephritis, dermatomyositis, myasthenia gravis, membranoproliferative glomerulonephritis (MPGN), psoriasis, multiple sclerosis, and injuries resulting from renal ischemia/reperfusion.

The present invention is illustrated in the figures and the examples provided below. These figures and examples are in no way intended to limit the scope of the present invention.

### FIGURES

FIG. 1 is a diagram representing the strategy for constructing the N-terminal fragments of the recombinant proteins according to the invention.

FIG. 2 is a diagram representing the strategy for constructing the C-terminal fragments of the recombinant proteins according to the invention.

FIGS. 3A, 3B and 3C are graphs showing the activity of acceleration of the dissociation of C3 convertase for the factor H fragments having the highest productivity.

### EXAMPLES

### Example 1

### Construction of N-ter and C-ter Fragments of Factor H in pCEP4 Plasmid

The goal is to subclone in various forms the N-terminal and C-terminal fragments of the Y402 variant of factor H in an optimized version in pCEP4 expression vector. Both fragments will be supplemented at the 5′ end with a NotI site, the Kozak sequence and a signal peptide, and at the 3′ end with a His-TAG followed by the BamHI site, the difference being a NheI site located at the 3′ end for the N-ter fragments and at the 5′ end for the C-ter fragments. Explanatory diagrams will be described in the protocol provided below.

I/Construction of pCEP4-N-ter Vectors

1/Construction of N-ter Fragments

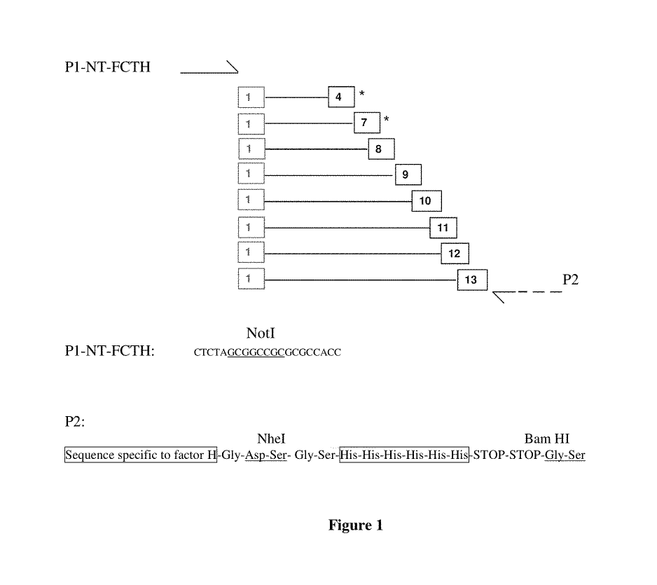

N-ter fragments are constructed by PCR from the pCDNA2001neo-MD3Y vector. This vector corresponds to the pCDNA2001neo vector containing the nucleic acid represented by the sequence SEQ ID NO: 90, which is an optimized sequence encoding the Y402 variant of factor H comprising an artificial signal peptide SP-MB7. The construction strategy is represented in FIG. 1.

The following primers are used:

sense Primer:

P1-NT-FCTH
(SEQ ID NO: 91)
5′-CTCTAGCGGCCGCGCGCCACC-3′

(nucleotides 6 to 13 of this sequence define a NotI restriction site)

Antisense Primers (or P2-NT-X Primers):

Each of these primers contains, in this order from 5′ to 3′: a BamHI site, two stop codons, a hexahistidine tag, a linker (GS), a NheI site and a sequence specific to factor H. These elements are represented in FIG. 1.

P2-NT-4
(SEQ ID NO: 92)
CTCTAGGATCCTTATCAATGGTGGTGATGGTGGTGGCTGCCGCTAGCGCC
AGATTTTTCCTCGCAGGAAGGCAG 75 bp
P2-NT-7
(SEQ ID NO: 93)
CTCTAGGATCCTTATCAATGGTGGTGATGGTGGTGGCTGCCGCTAGCGCC
AGTTTTCACGCGGATGCACCTTG 74 bp
P2-NT-8
(SEQ ID NO: 94)
CTCTAGGATCCTTATCAATGGTGGTGATGGTGGTGGCTGCCGCTAGCGCC
AGACTTGATGCAGGTAGGCTGG 73 bp
P2-NT-9
(SEQ ID NO: 95)
CTCTAGGATCCTTATCAATGGTGGTGATGGTGGTGGCTGCCGCTAGCGCC
TTCCCGCTCATAGCAGATGGGCAG 75 bp
P2-NT-10
(SEQ ID NO: 96)
CTCTAGGATCCTTATCAATGGTGGTGATGGTGGTGGCTGCCGCTAGCGCC
GCTCTGCACCTGCTCCTTGCAAATAG 77 bp
P2-NT-11
(SEQ ID NO: 97)
CTCTAGGATCCTTATCAATGGTGGTGATGGTGGTGGCTGCCGCTAGCGCC
GGTGGATTCCTCGACGATGCAC 73 bp
P2-NT-12
(SEQ ID NO: 98)
CTCTAGGATCCTTATCAATGGTGGTGATGGTGGTGGCTGCCGCTAGCGCC
TTTCTTCAGTTTGTCAATGGCGAC 75 bp
P2-NT-13
(SEQ ID NO: 99)
CTCTAGGATCCTTATCAATGGTGGTGATGGTGGTGGCTGCCGCTAGCGCC
CAGCTGGATCTGTGCCATAGAGCAG 76 bp

This P2-NT-X (X, variable) primer series is obtained by assembly PCR.
Assembly PCR between the P2NT primer and P2-X (X, variable) primers below:

It is carried out by means of the following primers:

1-P2NT
(SEQ ID NO: 100)
CTCTAGGATCCTTATCAATGGTGGTGATGGTGGTGGCTGCCGCTAGC
2-P2-4
(SEQ ID NO: 101)
CTGCCTTCCTGCGAGGAAAAATCTGGCGCTAGCGGCAGCCAC
2-P2-7
(SEQ ID NO: 102)
CAAGGTGCATCCGCGTGAAAACTGGCGCTAGCGGCAGCCAC
2-P2-8
(SEQ ID NO: 103)
CCAGCCTACCTGCATCAAGTCTGGCGCTAGCGGCAGCCAC
2-P2-9
(SEQ ID NO: 104)
CTGCCCATCTGCTATGAGCGGGAAGGCGCTAGCGGCAGCCAC
2-P2-10
(SEQ ID NO: 105)
TATTTGCAAGGAGCAGGTGCAGAGCGGCGCTAGCGGCAGCCAC
2-P2-11
(SEQ ID NO: 106)
GTGCATCGTCGAGGAATCCACCGGCGCTAGCGGCAGCCAC
2-P2-12
(SEQ ID NO: 107)
GTCGCCATTGACAAACTGAAGAAAGGCGCTAGCGGCAGCCAC
2-P2-13
(SEQ ID NO: 108)
CTGCTCTATGGCACAGATCCAGCTGGGCGCTAGCGGCAGCCAC

Amplification of the fragments of interest:

| | Antisense | Fragment of | | Amplicon |
|---|---|---|---|---|
| Sense primer | primer | interest | Matrix | size (bp) |
| P1-NT-FCTH | P2-NT-4 | Nter 1-4 | pCDNA2001neo-MD3Y | 865 |
| P1-NT-FCTH | P2-NT-7 | Nter 1-7 | pCDNA2001neo-MD3Y | 1408 |
| P1-NT-FCTH | P2-NT-8 | Nter 1-8 | pCDNA2001neo-MD3Y | 1567 |
| P1-NT-FCTH | P2-NT-9 | Nter 1-9 | pCDNA2001neo-MD3Y | 1747 |
| P1-NT-FCTH | P2-NT-10 | Nter 1-10 | pCDNA2001neo-MD3Y | 1930 |
| P1-NT-FCTH | P2-NT-11 | Nter 1-11 | pCDNA2001neo-MD3Y | 2113 |
| P1-NT-FCTH | P2-NT-12 | Nter 1-12 | pCDNA2001neo-MD3Y | 2299 |
| P1-NT-FCTH | P2-NT-13 | Nter 1-13 | pCDNA2001neo-MD3Y | 2473 |

2/Construction of Vectors
NotI/BamHI digestion of inserts:

| Inserts | Size (bp) |
|---|---|
| Nter 1-4 | 850 |
| Nter 1-7 | 1393 |
| Nter 1-8 | 1552 |
| Nter 1-9 | 1732 |
| Nter 1-10 | 1915 |
| Nter 1-11 | 2098 |
| Nter 1-12 | 2284 |
| Nter 1-13 | 2458 |

NotI/BamHI digestion of pCEP4 vector:
   →Two fragments of 24 and 10162 bp are obtained
      NucleoSpin Extract II (Clontech)
Ligation and transformation into TOP10 bacteria
PCR screening: CMV1/MD2-1Rev→A 594 bp amplicon is obtained
CMV1 primers—SEQ ID NO: 109: 5′-CCATTGACGTCAATGGGAGTTTG-3′
MD2-1rev—SEQ ID NO: 110: 5′-tgtcacactcgcggtagttg-3′

3/Sequencing

CMV1 and SV40-3′UTR primers are used for the sequencing of all the vectors. Only the vectors pCEP4-1NTer11, pCEP4-1NTer12 and pCEP4-1NTer13 have additional sequencing with MD2-3 primer.

SV40-3′UTR primer-SEQ ID NO: 111:
5′-TTCACTGCATTCTAGTTGTGGT-3′

II/ Construction of pCEP4-C-ter Vectors

1/Construct of C-ter Fragments

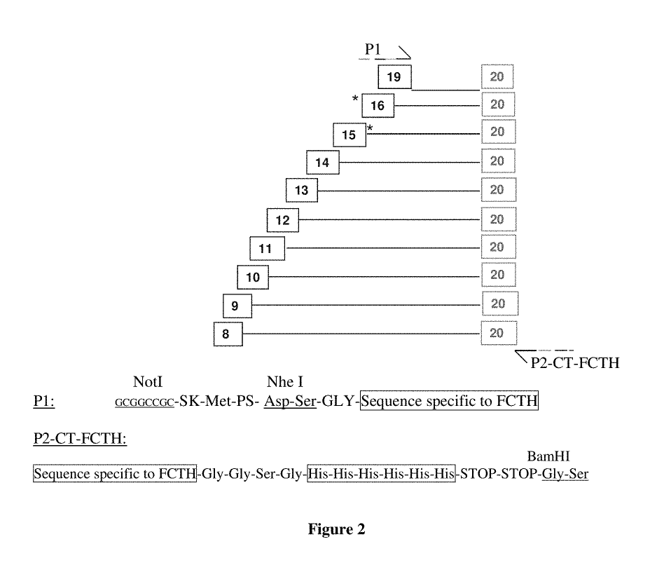

C-ter fragments are constructed by PCR from the pCDNA2001neo-MD3Y vector. This vector corresponds to the pCDNA2001neo vector containing the nucleic acid represented by the sequence SEQ ID NO: 90. The construction strategy is represented in FIG. 2.

The following primers are used:

sense primers (or P1-CT-X primers):

Each of these primers contains, in this order from 5′ to 3′: a NotI site, a Kozak sequence (KS), a signal peptide (SP), a NheI site and a sequence specific to factor H. These elements are represented in FIG. 2.

P1-CT-19
(SEQ ID NO: 112)
CTCTAGCGGCCGCGCGCCACCATGCGATGGTCTTGGATTTTTCTGCTGCT
GCTGTCTATCACTTCTGCTAACGCTGCTAGCGGCTGTGGACCCCCTCCAC
CCATC
P1-CT-16
(SEQ ID NO: 113)
CTCTAGCGGCCGCGCGCCACCATGCGATGGTCTTGGATTTTTCTGCTGCT
GCTGTCTATCACTTCTGCTAACGCTGCTAGCGGCTGTAAGAGTCCTCCAG
AGATTTCACA
P1-CT-15
(SEQ ID NO: 114)
CTCTAGCGGCCGCGCGCCACCATGCGATGGTCTTGGATTTTTCTGCTGCT
GCTGTCTATCACTTCTGCTAACGCTGCTAGCGGCTGTAGCCAGCCCCCTC
AGATC
P1-CT-14
(SEQ ID NO: 115)
CTCTAGCGGCCGCGCGCCACCATGCGATGGTCTTGGATTTTTCTGCTGCT
GCTGTCTATCACTTCTGCTAACGCTGCTAGCGGCTGCCCACCACCTCCAC
AGATTC
P1-CT-13
(SEQ ID NO: 116)
CTCTAGCGGCCGCGCGCCACCATGCGATGGTCTTGGATTTTTCTGCTGCT
GCTGTCTATCACTTCTGCTAACGCTGCTAGCGGCTGCAAGTCCTCTAATC
TGATCATTC
P1-CT-12
(SEQ ID NO: 117)
CTCTAGCGGCCGCGCGCCACCATGCGATGGTCTTGGATTTTTCTGCTGCT
GCTGTCTATCACTTCTGCTAACGCTGCTAGCGGCTGTGGCGATATTCCAG
AACTGG
P1-CT-11
(SEQ ID NO: 118)
CTCTAGCGGCCGCGCGCCACCATGCGATGGTCTTGGATTTTTCTGCTGCT
GCTGTCTATCACTTCTGCTAACGCTGCTAGCGGCTGTGGACCACCTCCAG
AACTGC
P1-CT-10
(SEQ ID NO: 119)
CTCTAGCGGCCGCGCGCCACCATGCGATGGTCTTGGATTTTTCTGCTGCT
GCTGTCTATCACTTCTGCTAACGCTGCTAGCGGCTGTGAGCTGCCAAAAA
TTGATG
P1-CT-9
(SEQ ID NO: 120)
CTCTAGCGGCCGCGCGCCACCATGCGATGGTCTTGGATTTTTCTGCTGCT
GCTGTCTATCACTTCTGCTAACGCTGCTAGCGGCTGTGACATTCCAGTGT
TTATGAACG
P1-CT-8
(SEQ ID NO: 121)
CTCTAGCGGCCGCGCGCCACCATGCGATGGTCTTGGATTTTTCTGCTGCT
GCTGTCTATCACTTCTGCTAACGCTGCTAGCGGCTGTTCTAAGAGCAGCA
TCGACATTG

antisense primer (P2-CT-FCTH)

This primer contains, in the 5′ to 3′ direction: a BamHI site, two stop codons, a hexahistidine tag, a linker (GGSG) and a specific sequence of factor H

P2-CT-FCTH
(SEQ ID NO: 122)
GAGTAGGATCCTTATCAATGGTGGTGATGGTGGTGGCCGCTTCCGCCTCT
CTTAGCACAAGTAGGGTATTCCAG 74 bp

The P1-CT-X (X, variable) primer series and the P2-CT-FCTH primer are obtained by assembly PCR.
Assembly PCR to obtain the primers:

primers used to obtain the P2-CT-FCTH primer

1-P2-CT-FCTH-for
(SEQ ID NO: 123)
GAGTAGGATCCTTATCAATGGTGGTGATGGTGGTGGCCGCTTCCG
2-P2-CT-FCTH-rev
(SEQ ID NO: 124)
CTGGAATACCCTACTTGTGCTAAGAGAGGCGGAAGCGGCCACCAC

primers used to obtain the P1-CT-X primers

PCR1:

1-P1-CT-FCTH1
(SEQ ID NO: 125)
CTCTAGCGGCCGCGCGCCACCATGCGATGGTCTTG
1-P1-CT-FCTH2
(SEQ ID NO: 126)
CGTTAGCAGAAGTGATAGACAGCAGCAGCAGAAAAATCCAAGACCATCGC
ATG
Fragment PCR1: P1-CT-FCTH-PCR1
(SEQ ID NO: 127)
CTCTAGCGGCCGCGCGCCACCATGCGATGGTCTTGGATTTTTCTGCTGCT
GCTGTCTATCACTTCTGCTAACG 73 bp

| Sense | Antisense | Dimer | Dimer |
|---|---|---|---|
| primer | primer | created | size (bp) |
| 1-P1-CT-FCTH1 | 1-P1-CT-FCTH2 | P1-CT-FCTH-Pcr1 | 73 |

PCR2:

2-P1-CT-19
(SEQ ID NO: 128)
GATGGGTGGAGGGGGTCCACAGCCGCTAGCAGCGTTAGCAGAAGTGA
2-P1-CT-16
(SEQ ID NO: 129)
TGTGAAATCTCTGGAGGACTCTTACAGCCGCTAGCAGCGTTAGCAGAAGT
GA
2-P1-CT-15
(SEQ ID NO: 130)
GATCTGAGGGGGCTGGCTACAGCCGCTAGCAGCGTTAGCAGAAGTGA
2-P1-CT-14
(SEQ ID NO: 131)
GAATCTGTGGAGGTGGTGGGCAGCCGCTAGCAGCGTTAGCAGAAGTGA
2-P1-CT-13
(SEQ ID NO: 132)
GAATGATCAGATTAGAGGACTTGCAGCCGCTAGCAGCGTTAGCAGAAGT
GA
2-P1-CT-12
(SEQ ID NO: 133)
CCAGTTCTGGAATATCGCCACAGCCGCTAGCAGCGTTAGCAGAAGTGA
2-P1-CT-11
(SEQ ID NO: 134)
GCAGTTCTGGAGGTGGTCCACAGCCGCTAGCAGCGTTAGCAGAAGTGA
2-P1-CT-10
(SEQ ID NO: 135)
CATCAATTTTTGGCAGCTCACAGCCGCTAGCAGCGTTAGCAGAAGTGA
2-P1-CT-9
(SEQ ID NO: 136)
CGTTCATAAACACTGGAATGTCACAGCCGCTAGCAGCGTTAGCAGAAGT
GA
2-P1-CT-8
(SEQ ID NO: 137)
CAATGTCGATGCTGCTCTTAGAACAGCCGCTAGCAGCGTTAGCAGAAGT
GA

| | Antisense | Dimer | Dimer |
|---|---|---|---|
| Fragment | primer | created | size (bp) |
| P1-CT-FCTH-Pcr1 | 2-P1-CT-19 | P1-CT-19 | 106 |
| P1-CT-FCTH-Pcr1 | 2-P1-CT-16 | P1-CT-16 | 111 |
| P1-CT-FCTH-Pcr1 | 2-P1-CT-15 | P1-CT-15 | 106 |
| P1-CT-FCTH-Pcr1 | 2-P1-CT-14 | P1-CT-14 | 107 |
| P1-CT-FCTH-Pcr1 | 2-P1-CT-13 | P1-CT-13 | 110 |
| P1-CT-FCTH-Pcr1 | 2-P1-CT-12 | P1-CT-12 | 107 |
| P1-CT-FCTH-Pcr1 | 2-P1-CT-11 | P1-CT-11 | 107 |
| P1-CT-FCTH-Pcr1 | 2-P1-CT-10 | P1-CT-10 | 107 |
| P1-CT-FCTH-Pcr1 | 2-P1-CT-9 | P1-CT-9 | 110 |
| P1-CT-FCTH-Pcr1 | 2-P1-CT-8 | P1-CT-8 | 110 |

Amplification of the fragments of interest:

| Sense | Antisense | Fragment of | | Amplicon |
|---|---|---|---|---|
| primers | primers | interest | Matrix | size (bp) |
| P1-CT-19 | P2-CT-FCTH | Cter 19-20 | pCDNA2001neo- | 500 |
| | | | MD3Y | |
| P1-CT-16 | P2-CT-FCTH | Cter 16-20 | pCDNA2001neo- | 1034 |
| | | | MD3Y | |
| P1-CT-15 | P2-CT-FCTH | Cter 15-20 | pCDNA2001neo- | 1217 |
| | | | MD3Y | |
| P1-CT-14 | P2-CT-FCTH | Cter 14-20 | pCDNA2001neo- | 1394 |
| | | | MD3Y | |
| P1-CT-13 | P2-CT-FCTH | Cter 13-20 | pCDNA2001neo- | 1568 |
| | | | MD3Y | |
| P1-CT-12 | P2-CT-FCTH | Cter 12-20 | pCDNA2001neo- | 1754 |
| | | | MD3Y | |
| P1-CT-11 | P2-CT-FCTH | Cter 11-20 | pCDNA2001neo- | 1937 |
| | | | MD3Y | |
| P1-CT-10 | P2-CT-FCTH | Cter 10-20 | pCDNA2001neo- | 2120 |
| | | | MD3Y | |
| P1-CT-9 | P2-CT-FCTH | Cter 9-20 | pCDNA2001neo- | 2300 |
| | | | MD3Y | |
| P1-CT-8 | P2-CT-FCTH | Cter 8-20 | pCDNA2001neo- | 2483 |
| | | | MD3Y | |

2/Construction of Vectors
NotI/BamHI digestion of inserts:

| Inserts | Size (bp) |
|---|---|
| Cter 19-20 | 483 |
| Cter 16-20 | 1017 |
| Cter 15-20 | 1200 |
| Cter 14-20 | 1377 |
| Cter 13-20 | 1551 |
| Cter 12-20 | 1737 |
| Cter 11-20 | 1920 |
| Cter 10-20 | 2110 |
| Cter 9-20 | 2283 |
| Cter 8-20 | 2466 |

NotI/BamHI digestion of pCEP4 vector:
   →Two fragments of 24 and 10162 bp are obtained
      NucleoSpin Extract II
Ligation and transformation of TOP10 bacteria
PCR screening: CMV1/P2-MB7→A 259 bp amplicon is obtained

Seq P2-MB7-SEQ ID NO: 138:
5′-TGGTGATGCTCAGCAGCAGCAGGAAGATCCAGCTCCATCG-3′

3/Sequencing

CMV1 and SV40-3′UTR primers are used for the sequencing of all the vectors. Only the pCEP4-9Cter20 and pCEP4-8Cter20 vectors will have supplemental sequencing with MD2- 5 primer.

Seq MD2-5-SEQ ID NO: 139:
5′-GGATTCACACCGTGTGCATTAATG-3′

### Example 2

### Construction of Factor H Vectors Combining the N-ter and C-ter domains

From the 8 pCEP4-1NTX vectors and the 10 pCEP4-XCT20 vectors we construct 59 vectors combining the N-ter and C-ter fragments, containing obligatorily at least the SCR1-4 N-terminal domains and the SCR19-20 C-terminal domains to which are added a variable number of SCR domains in the central portion of the molecule.

First, we introduce the 1NTX fragments present in the pCEP4 plasmid into the pCDNA2001neo vector (by NotI/BamHI digestion).

Second, we introduce into the pCDNA2001neo-1NTX vectors the XCT20 fragments (by NheI/BamHI digestion).

We thus obtain 59 plasmid constructs which correspond to the 59 1NTX-XCT20 combinations of the FH fragments. These sequences are present in the pCDNA2001neo vector, which permits stable expression of these molecules in the PER.C6 cell line. To facilitate the screening and production work, 59 FH 1NTX-XCT20 fragments are extracted from the pCDNA2001neo vector by NotI/BamHI digestion and cloned into pCEP4 vector, which permits transient expression in HEK293F cells.

I/Construction of pCDNA2001neo-N-Ter vectors from pCEP4-N-ter Vectors:
-Digestion of pCEP4-1NT X vectors by NotI/BamHI:

Gel purification of the fragment of interest followed by NucleoSpin Extract II.
Digestion of pCDNA2001neo vector by NotI/BamHI.

NucleoSpin Extract II.
Ligation and TOP10 transformation.
PCR screening: CMV1/MD2-1REV: A 706 bp fragment is obtained.
NotI/BamHI digestion control: confirmation of the presence of the insert.

II/ Construction of pCDNA2001neo-1NTX / XCT20 vectors for Production in the PER.C6 Cell Line:

| Nter | Cter |
|---|---|
| fragment | fragment |
| 1NT4 | 19CT20 |
| | 16CT20 |
| | 15CT20 |
| | 14CT20 |
| | 13CT20 |
| | 12CT20 |
| | 11CT20 |
| | 10CT20 |
| | 9CT20 |
| | 8CT20 |
| 1NT7 | 19CT20 |
| | 16CT20 |
| | 15CT20 |
| | 14CT20 |
| | 13CT20 |
| | 12CT20 |
| | 11CT20 |
| | 10CT20 |
| | 9CT20 |
| | 8CT20 |
| 1NT8 | 19CT20 |
| | 16CT20 |
| | 15CT20 |
| | 14CT20 |
| | 13CT20 |
| | 12CT20 |
| | 11CT20 |
| | 10CT20 |
| | 9CT20 |
| 1NT9 | 19CT20 |
| | 16CT20 |
| | 15CT20 |
| | 14CT20 |
| | 13CT20 |
| | 12CT20 |
| | 11CT20 |
| | 10CT20 |
| 1NT10 | 19CT20 |
| | 16CT20 |
| | 15CT20 |
| | 14CT20 |
| | 13CT20 |
| | 12CT20 |
| | 11CT20 |
| 1NT11 | 19CT20 |
| | 16CT20 |
| | 15CT20 |
| | 14CT20 |
| | 13CT20 |
| | 12CT20 |
| 1NT12 | 19CT20 |
| | 16CT20 |
| | 15CT20 |
| | 14CT20 |
| | 13CT20 |
| 1NT13 | 19CT20 |
| | 16CT20 |
| | 15CT20 |
| | 14CT20 |

Digestion of pCEP4-XCT20 vectors by NheI/BamHI:

| | |
|---|---|
| pCEP4-19CT20 | 414 bp |
| pCEP4-16CT20 | 948 bp |
| pCEP4-15CT20 | 1131 bp |
| pCEP4-14CT20 | 1308 bp |
| pCEP4-13CT20 | 1482 bp |
| pCEP4-12CT20 | 1668 bp |
| pCEP4-11CT20 | 1851 bp |
| pCEP4-10CT20 | 2034 bp |
| pCEP4-9CT20 | 2214 bp |
| pCEP4-8CT20 | 2397 bp |

Gel purification of the fragment of interest followed by NucleoSpin Extract II.
Digestion of pCDNA2001neo vector-1NTX by NheI/BamHI.

NucleoSpin Extract II.
Ligation and TOP10 transformation.
PCR screening: MD2-6/2BGHPA: A 373 bp fragment is obtained.

NotI/BamHI and NheI/BamHI digestion control.

Seq MD2-6-SEQ ID NO: 140:
5′-AGGGAAACAAGCGCATCACCT-3′
Seq 2BGHPA-SEQ ID NO: 141:
5′-CAGATGGCTGGCAACTAGAA-3′

The 1NTX/XCT20 fragments are then extracted by NotI/BamHI digestion and reintroduced into pCEP4 vector.

III/ Construction of pCEP4-1NTX/XCT20 Vectors for Production in the HEK 293 Freestyle Cell Line:
Digestion of pCEP4 vector by NotI/BamHI (10162 and 24 bp)
   NucleoSpin Extract II.
Digestion of pCDNA2001neo-1NTX/XCT20 vectors by NotI/BamHI (fragment size in the table below.)
   Gel purification
   NucleoSpin Extract II.

| | Insert |
|---|---|
| Digested vector (NotI/BamHI) | size (bp) |
| pCDNA2001neo-1NT4/19CT20 | 1216 |
| pCDNA2001neo-1NT4/16CT20 | 1762 |
| pCDNA2001neo-1NT4/15CT20 | 1945 |
| pCDNA2001neo-1NT4/14CT20 | 2122 |
| pCDNA2001neo-1NT4/13CT20 | 2296 |
| pCDNA2001neo-1NT4/12CT20 | 2482 |
| pCDNA2001neo-1NT4/11CT20 | 2665 |
| pCDNA2001neo-1NT4/10CT20 | 2848 |
| pCDNA2001neo-1NT4/9CT20 | 3028 |
| pCDNA2001neo-1NT4/8CT20 | 3211 |
| pCDNA2001neo-1NT7/19CT20 | 1771 |
| pCDNA2001neo-1NT7/16CT20 | 2305 |
| pCDNA2001neo-1NT7/15CT20 | 2488 |
| pCDNA2001neo-1NT7/14CT20 | 2665 |
| pCDNA2001neo-1NT7/13CT20 | 2839 |
| pCDNA2001neo-1NT7/12CT20 | 3025 |
| pCDNA2001neo-1NT7/11CT20 | 3208 |
| pCDNA2001neo-1NT7/10CT20 | 3391 |
| pCDNA2001neo-1NT7/9CT20 | 3571 |
| pCDNA2001neo-1NT7/8CT20 | 3754 |
| pCDNA2001neo-1NT8/19CT20 | 1954 |
| pCDNA2001neo-1NT8/16CT20 | 2488 |
| pCDNA2001neo-1NT8/15CT20 | 2671 |
| pCDNA2001neo-1NT8/14CT20 | 2848 |
| pCDNA2001neo-1NT8/13CT20 | 3022 |
| pCDNA2001neo-1NT8/12CT20 | 3208 |
| pCDNA2001neo-1NT8/11CT20 | 3391 |
| pCDNA2001neo-1NT8/10CT20 | 3574 |
| pCDNA2001neo-1NT8/9CT20 | 3754 |
| pCDNA2001neo-1NT9/19CT20 | 2134 |
| pCDNA2001neo-1NT9/16CT20 | 2668 |
| pCDNA2001neo-1NT9/15CT20 | 2851 |
| pCDNA2001neo-1NT9/14CT20 | 3028 |
| pCDNA2001neo-1NT9/13CT20 | 3202 |
| pCDNA2001neo-1NT9/12CT20 | 3388 |
| pCDNA2001neo-1NT9/11CT20 | 3571 |
| pCDNA2001neo-1NT9/10CT20 | 3754 |
| pCDNA2001neo-1NT10/19CT20 | 2317 |
| pCDNA2001neo-1NT10/16CT20 | 2851 |
| pCDNA2001neo-1NT10/15CT20 | 3034 |
| pCDNA2001neo-1NT10/14CT20 | 3211 |
| pCDNA2001neo-1NT10/13CT20 | 3385 |
| pCDNA2001neo-1NT10/12CT20 | 3571 |
| pCDNA2001neo-1NT10/11CT20 | 3754 |
| pCDNA2001neo-1NT11/19CT20 | 2500 |
| pCDNA2001neo-1NT11/16CT20 | 3034 |
| pCDNA2001neo-1NT11/15CT20 | 3217 |
| pCDNA2001neo-1NT11/14CT20 | 3394 |
| pCDNA2001neo-1NT11/13CT20 | 3568 |
| pCDNA2001neo-1NT11/12CT20 | 3754 |
| pCDNA2001neo-1NT12/19CT20 | 2686 |
| pCDNA2001neo-1NT12/16CT20 | 3220 |
| pCDNA2001neo-1NT12/15CT20 | 3403 |
| pCDNA2001neo-1NT12/14CT20 | 3580 |
| pCDNA2001neo-1NT12/13CT20 | 3754 |
| pCDNA2001neo-1NT13/19CT20 | 2860 |
| pCDNA2001neo-1NT13/16CT20 | 3394 |
| pCDNA2001neo-1NT13/15CT20 | 3577 |
| pCDNA2001neo-1NT13/14CT20 | 3754 |

Ligation and TOP10 transformation.
PCR screening: CMV1/MD2-1REV: A 594 bp fragment is obtained.
Junction sequencing by CMV1 and SV40-3′UTR.

### Example 3

### Determination of the Concentration and the Molecular Mass of FH Fragments Present in the Supernatants of HEK 293F Cells after 7 Days of Production in Batch Mode

### 3.1: Transient Transfection into HEK 293F Cells for Transient Production of Recombinant FH Fragments

1-Transient transfection:

The day before the transient transfection, HEK 293F cells are subcultured at a cell concentration of 7^{E}5 vc/ml. The cell density and the viability of the HEK 293F cells are measured the day of the transfection. A volume of culture corresponding to 30^{E}6 cv/ml is centrifuged. The supernatant is discarded and the cell pellet is taken up in 28 ml of F17 culture medium (Invitrogen), transferred to a 250 ml Erlenmeyer flask and incubated at 37° C.

2-Formation of the transfection agent / DNA complex in a 2:1 ratio:

The transfection agent and the DNA corresponding to the pCEP4 vector containing one of the FH fragment sequences are prepared in OptiMEM medium (Invitrogen) as follows:
Addition of 30 μg of DNA in 1 ml of OptiMEM
Addition of 60 μl of Fectin or 60 μl of PEI in 1 ml of OptiMEM.

These two preparations are incubated separately for 5 minutes at room temperature and then the solution containing the transfection agent is added delicately to that containing the DNA. The mixture is incubated at room temperature for 25 minutes before being added to the 28 ml of HEK 293F cells prepared beforehand. The cells are then incubated at 37° C. with shaking at 125 rpm.

3-Transient production of factor H:

The cells are maintained in culture for 7 days with no addition or refreshing of culture medium. On the 7^{th} day, the cells are centrifuged at 3000 g for 15 minutes. The cell pellets are discarded and the cell supernatants containing the recombinant FH fragments are filtered through a 0.22 μm filter and then frozen at −20° C.

### 3.2: Assay of Human FH Fragments in the Culture Medium by ELISA

Coating antibody: Sheep anti-human factor H immunoglobulin (The Binding Site) freshly diluted in pH 7.4 PBS buffer so as to obtain a concentration of 3.5 to 5.5 μg/ml.

Saturation buffer: PBS—1% BSA (w/v)

Washing buffer: PBS—0.1% Tween-20 (v/v)

Dilution buffer: PBS—0.1% Tween-20 (v/v)—0.1% BSA (w/v)

Standard solution: Human factor H calibrator NL (The Binding Site)

Samples: The samples are prediluted so as to obtain a concentration close to that of the first point of the range. Then dilute ½ in ½.

Anti-factor H monoclonal antibody: Purified mouse monoclonal anti-human factor H immunoglobulins (SEROTEC)

Detection antibody: Goat anti-mouse IgG immunoglobulins conjugated with peroxidase (Jackson Immuno Research Laboratories).

Detection solution: TMB Kit (Pierce)

Stop solution: 4 N or 2 M sulfuric acid (Fisher Scientific).

Procedure

Sensitization of the solid phase:

In a microtiter plate, 100 μl of diluted coating antibody is distributed per well. The plate is then covered with adhesive film and incubated at +4° C. overnight. The plate is then emptied by inverting it.

Saturation of the solid phase:

120 μl of saturation solution is distributed per well. The plate is then covered with adhesive film and incubated at 20° C. for 1 hour. Washing is then carried out 3 times with washing buffer.

Antigen Capture:

100 μl of dilution buffer (blank), of each dilution of the standard and of the samples is distributed per well. The plate is then covered with adhesive film and incubated at 20° C. for 1 hour 30 minutes. Washing is then carried out 5 times with washing buffer.

Recognition of the antigen by the monoclonal antibody:

100 μl of the monoclonal antibody is distributed in each well. The plate is covered with adhesive film and then incubated at 20° C. for 1 hour 30 minutes. Washing is then carried out 5 times with washing buffer.

Labeling with horseradish peroxidase (HRP) conjugate:

100 μl of detection antibody is distributed, the plate is covered with adhesive film and then incubated at 20° C. for 1 hour. The wells are washed 5 times with washing buffer.

Enzymatic reaction: 100 μl per well of TMB containing the substrate is distributed at regular time intervals and away from any intense light. Incubation is then carried out at room temperature for 5 to 15 minutes. This time must be identical for all the assay points.

Stopping the reaction: 100 μl of 4N H₂SO₄ per well is distributed at regular time intervals.

The results are reported in the following table.

| | ELISA (batch | Molecular |
|---|---|---|
| | mode, 7 days) | mass |
| FH fragment | mg/L | (Daltons) |
| 1NT4-19CT20 | 67 | 43442.8 |
| 1NT4-16CT20 | 46 | 63288.2 |
| 1NT4-15CT20 | 29 | 69983.5 |
| 1NT4-14CT20 | 15 | 76680.1 |
| 1NT4-11CT20 | 10 | 97371.7 |
| 1NT4-10CT20 | 22 | 104213.6 |
| 1NT4-9CT20 | 13 | 111090.2 |
| 1NT4-8CT20 | 15 | 117650.5 |
| 1NT7-19CT20 | 10 | 64198.2 |
| 1NT7-16CT20 | 76 | 84043.6 |
| 1NT7-15CT20 | 132 | 90738.9 |
| 1NT7-14CT20 | 177 | 97435.5 |
| 1NT7-13CT20 | 27 | 104274.3 |
| 1NT7-12CT20 | 42 | 111244.2 |
| 1NT7-11CT20 | 154 | 118127.1 |
| 1NT7-10CT20 | 157 | 124969.1 |
| 1NT7-9CT20 | 161 | 131845.6 |
| 1NT7-8CT20 | 224 | 138405.9 |
| 1NT8-19CT20 | 7 | 70758.5 |
| 1NT8-16CT20 | 47 | 90603.9 |
| 1NT8-15CT20 | 124 | 97299.2 |
| 1NT8-14CT20 | 339 | 103969.8 |
| 1NT8-13CT20 | 48 | 110808.6 |
| 1NT8-12CT20 | 13 | 117804.6 |
| 1NT8-11CT20 | 115 | 124687.4 |
| 1NT8-10CT20 | 128 | 131529.4 |
| 1NT8-9CT20 | 80 | 138405.9 |
| 1NT9-19CT20 | 5 | 77635.1 |
| 1NT9-16CT20 | 8 | 97480.4 |
| 1NT9-15CT20 | 43 | 104175.7 |
| 1NT9-14CT20 | 91 | 110872.3 |
| 1NT9-13CT20 | 25 | 117711.2 |
| 1NT9-12CT20 | 25 | 124655.0 |
| 1NT9-11CT20 | 45 | 131537.9 |
| 1NT9-10CT20 | 156 | 138405.9 |
| 1NT10-19CT20 | 5 | 84451.0 |
| 1NT10-15CT20 | 13 | 110991.7 |
| 1NT10-14CT20 | 21 | 117688.3 |
| 1NT10-11CT20 | 128 | 138379.9 |
| 1NT11-19CT20 | 9 | 91333.8 |
| 1NT11-16CT20 | 22 | 111179.2 |
| 1NT11-15CT20 | 16 | 117874.5 |
| 1NT11-14CT20 | 36 | 124571.1 |
| 1NT13-14CT20 | 13 | 138379.9 |

### Example 4

### Characterization by SDS-PAGE of the Recombinant FH Fragments Present in the Culture Supernatant (FIGS. 6A, 6B and 7).

Based on the value of the concentration of recombinant FH fragments determined by ELISA, a volume corresponding to 1 μg of recombinant factor H is diluted to ½ in 2× Laemmli buffer, heated at 95° C. for 5 minutes and then deposited on a 10% polyacrylamide linear gel. After migration, the proteins contained in the gel are stained with Coomassie blue.

The analysis of the polyacrylamide gels shows that the recombinant FH fragments migrate according to the expected apparent molecular masses.

### Example 5

### Determination of the Activity of Acceleration of the Dissociation of C3 Convertase of the Recombinant FH Fragments Present in the Culture Supernatant and Having the Highest Productivity

Based on the concentration of recombinant factor H determined by ELISA, the recombinant FH fragments are diluted to a final concentration of 20 μg/ml. From this tube, a range is prepared by the following successive dilutions: ½; 1/10; ¼; ¼; ¼; ¼; 1/40. This range of decreasing concentration of FH fragments is added to the C3 convertase complex formed in the wells of a 96-well plate, which is then incubated at +34° C. for 32 to 34 minutes. After washing several times, the factor B still complexed with the C3 molecule immobilized at the bottom of the well is then assayed by an ELISA-type immunoenzymatic reaction. The absorbance values obtained as a function of the concentration of FH fragments added are then processed in a nonlinear modeling system (sigmoid of variable slope) in order to determine the IC₅₀ value of each sample. The equation for calculating this model is as follows:
Y: Bottom +(Top-Bottom)/(1+10̂((Log IC₅₀−X) * Hillslope)
X is the logarithm of the concentration (μg/ml).

Y is the response (OD).
Y starts at the baseline (Bottom) and goes to the top (Top) in a sigmoidal shape. Hillslope is the slope.

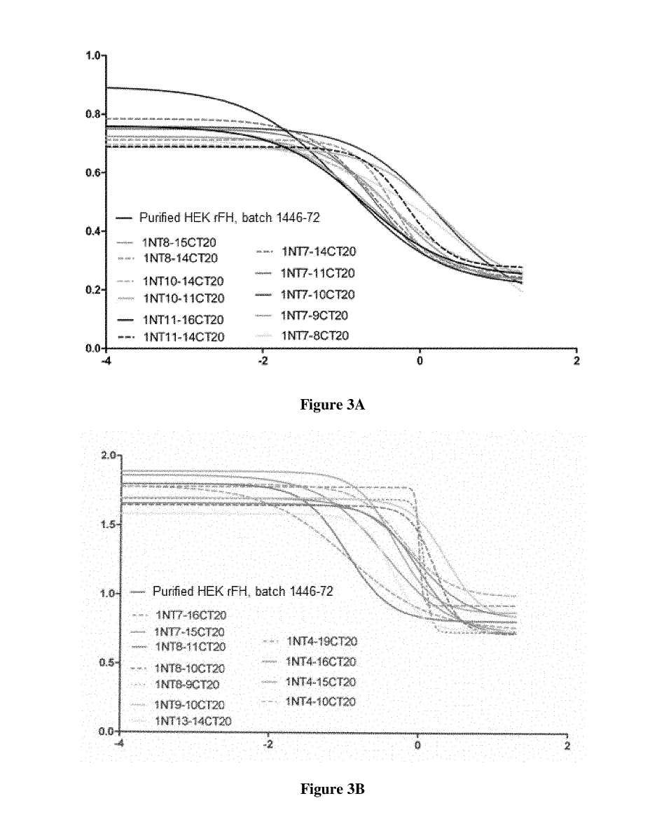

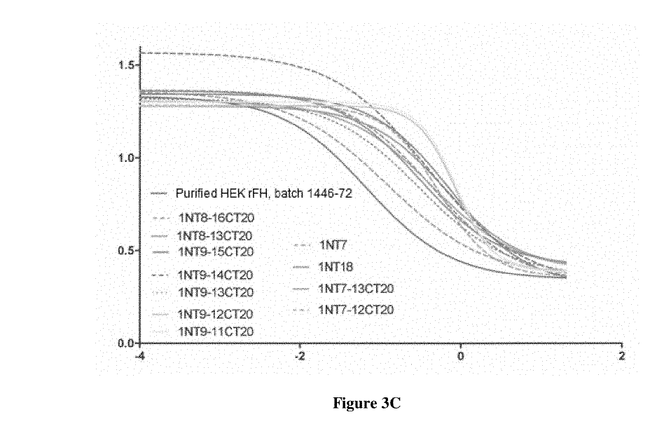

The activity determined in the cell supernatant is shown in FIGS. 3A, 3B and 3C for the fragments having the highest productivity.

### Example 6

### Determination of the Activity of Acceleration of the Dissociation of C3 Convertase of Purified Recombinant FH Fragments

The FH fragments were purified by means of the hexahistidine tag which was added at the C-terminus of the proteins. The purification was carried out in one step of affinity chromatography using HisTALON columns (Clontech) containing a resin having a strong affinity and specificity for polyhistidines. The purification was carried out according to the supplier's recommendations (HisTALON Gravity Column Purification Kit User Manual).
After purification the samples are dialyzed against PBS and stored at 4° C.

The concentration of the purified recombinant FH fragments is determined by measuring OD at 280 nm. The purified recombinant FH fragments are diluted to a final concentration of 150 nM and then from this tube a range is prepared by the following successive dilutions: ½; 1/10; ¼; ¼; ¼; ¼; 1/40. This range of decreasing concentration of FH fragments is added to the C3 convertase complex formed in the wells of a 96-well plate, which is then incubated at +34° C. for 32 to 34 minutes. After washing several times, the factor B still complexed with the C3 molecule immobilized at the bottom of the well is then assayed by an ELISA-type immunoenzymatic reaction using 3,3′,5,5′-tetramethylbenzidine (TMB) or ortho-phenylenediamine (OPD) as reaction substrate. The absorbance values obtained as a function of the concentration of added FH fragments are then processed in a nonlinear modeling system (sigmoid of variable slope) in order to determine the IC₅₀ value of each sample. The equation for calculating this model is as follows:
Y: Bottom+(Top-Bottom)/(1+10̂((Log IC₅₀−X) * Hillslope)
X is the logarithm of the concentration (μg/ml).
Y is the response (OD).
Y starts at the baseline (Bottom) and goes to the top (Top) in a sigmoidal shape. Hillslope is the slope.

The IC₅₀ values obtained are indicated in the table below.

| | C3 | C3 |
|---|---|---|
| | convertase | convertase |
| | activity | activity |
| | IC₅₀ (μg/ml) | % of the control |
| pCEP4-1NT4-16CT20 | 0.0281 | 239% |
| pCEP4-1NT7-9CT20 | 0.2517 | 156% |
| pCEP4-1NT7-11CT20 | 0.2653 | 134% |
| pCEP4-1NT8-10CT20 | 0.3379 | 107% |
| pCEP4-1NT7-14CT20 | 0.0645 | 104% |
| pCEP4-1NT8-11CT20 | 0.4713 | 77% |
| pCEP4-1NT4-19CT20 | 0.0916 | 73% |
| pCEP4-1NT7-8CT20 | 0.5419 | 72% |
| pCEP4-1NT9-11CT20 | 0.1716 | 69% |
| pCEP4-1NT9-10CT20 | 0.5517 | 64% |
| pCEP4-1NT9-14CT20 | 0.5698 | 64% |
| pCEP4-1NT8-14CT20 | 0.6454 | 61% |
| pCEP4-1NT7-10CT20 | 0.6015 | 59% |
| pCEP4-1NT10-11CT20 | 0.6118 | 58% |
| pCEP4-1NT8-15CT20 | 0.6279 | 58% |
| pCEP4-1NT4-15CT20 | 0.2061 | 58% |
| pCEP4-1NT9-13CT20 | 0.1663 | 56% |
| pCEP4-1NT7-15CT20 | 0.6393 | 56% |
| pCEP4-1NT9-19CT20 | 0.2166 | 49% |
| pCEP4-1NT13-14CT20 | 0.7765 | 45% |
| pCEP4-1NT4-8CT20 | 0.2276 | 42% |
| pCEP4-1NT4-10CT20 | 0.2439 | 38% |
| pCEP4-1NT9-12CT20 | 0.3070 | 30% |
| pCEP4-1NT8-13CT20 | 0.4022 | 30% |
| pCEP4-1NT8-9CT20 | 0.4886 | 27% |
| pCEP4-1NT9-16CT20 | 0.4177 | 25% |
| pCEP4-1NT8-16CT20 | 0.4264 | 25% |
| pCEP4-1NT4-9CT20 | 0.5360 | 18% |
| pCEP4-1NT4-11CT20 | 0.5458 | 17% |
| pCEP4-1NT4-14CT20 | 0.6179 | 15% |
| pCEP4-1NT7-16CT20 | 0.4418 | 15% |
| pCEP4-1NT10-14CT20 | 0.6714 | 14% |
| pCEP4-1NT7-19CT20 | 1.0900 | 12% |
| pCEP4-1NT11-15CT20 | 0.7395 | 10% |
| pCEP4-1NT11-16CT20 | 1.2120 | 8% |
| pCEP4-1NT7-13CT20 | 1.9260 | 7% |
| pCEP4-1NT7-12CT20 | 2.0350 | 7% |
| pCEP4-1NT10-15CT20 | 1.1680 | 6% |
| pCEP4-1NT11-14CT20 | 1.9900 | 6% |
| pCEP4-1NT11-19CT20 | 1.7800 | 5% |
| pCEP4-1NT9-15CT20 | 2.1880 | 5% |
| pCEP4-1NT8-12CT20 | 1.8700 | 4% |
| pCEP4-1NT10-19CT20 | 6.0520 | 1% |
| pCEP4-1NT8-19CT20 | 493.0000 | 0% |

The activity of whole recombinant factor H produced in PER.C6 or HEK cells and purified was used as the activity control. The C3 activity of whole factor H produced in PER.C6 cells is equivalent to that of whole factor H produced in HEK cells.

### Example 7

### Determination of the Activity of Protection of the Lysis of Sheep Red Blood Cells Using a Modified Sanchez-Corral Test

This test makes it possible to measure the functional activity of the C-terminal portion of the recombinant human factor H purified during the development of therapeutic batches by evaluating its ability to protect sheep red blood cells from lysis induced by a serum depleted or deficient in functional factor H. This test is adapted from the “Sanchez-Corral” method for measuring the anti-hemolytic activity of the factor H present in the plasma of patients with hemolytic uremic syndrome (P. Sanchez-Corral, C. Gonzàlez-Rubio, S. Rodriguez de Cordoba and M. Lopez-Trascasa. Molecular Immunology 41 (2004) 81-84).

In this context, a mixture of serum depleted of factor H and of a human plasma pool is prepared in equal proportions in order to create the conditions of a specific lysis. The addition of purified recombinant human factor H provides protection of the sheep red blood cells (absence of cell lysis) against lysis induced by complement.

This test was used here to determine the activity of protection of the lysis of red blood cells of the factor H fragments according to the invention.

Determination of percentage lysis:

To 40 μl of a suspension of sheep red corpuscles (1×10⁸ red corpuscles/ml) is added successively 20 μl of reaction buffer (10 mM HEPES, 144 mM NaCl, 7 mM MgCl₂, 10 mM EGTA, pH 7.2), a variable volume of FH fragment or of whole FH (0-30 μl) corresponding to concentrations of 0 to 44.74 pM, and then 9 μl of a human plasma pool followed by 9 μl of human plasma depleted in FH. The reaction volume is supplemented with PBS so as to obtain a final volume of 110 μl. After incubation for 30 min at 37° C., 400 μl of cold HBS-EDTA buffer (10 mM HEPES, 144 mM NaC1, 2 mM EDTA, pH 7.2) is used to stop the reaction. After centrifugation for 5 min at 1730 g, 200 μl of supernatant is taken and deposited in a microtiter plate in order to measure absorbance at 414 nm.

The percentage lysis is determined according to the formula:

$$( \frac{{OD}_{414 n m} Reaction tube - {OD}_{414 n m} Blank tube}{{OD}_{414 n m 100 \%} lysis} ) \times 100$$

The “100% lysis” control corresponds to the maximum lysis of the sheep red blood cells observed in the presence of water. The blank control comprises the reaction buffer+50 mM EDTA and corresponds to spontaneous lysis of the sheep red blood cells.

Without CFH, spontaneous lysis of the red corpuscles is about 30%.

In the following tables, the analysis is made by normalizing the value obtained to 100% plasma factor H (FH LP03 0 pm). The negative values obtained are normalized to the value 0%.

| | | | | |
|---|---|---|---|---|
| | Control | | | pCEP4-1NT7- |
| Purified | LP03 | PER.C6 | HEK | 8CT20 |

| | |
|---|---|
| FH (10⁻¹² mol) | Specific lysis (%) |

| | | | | |
|---|---|---|---|---|
| 0 | 100 | 85.4 | 85.4 | 80.6 |
| 14.58 | 75 | 49.1 | 42.1 | 1.6 |
| 21.87 | 49.9 | 10.7 | 2.3 | 4.7 |
| 29.16 | 21.2 | 0.5 | 0.2 | 0 |
| 44.74 | 2.9 | 0 | 0.7 | 0 |

| | Control | | pCEP4-1NT7- | pCEP4-1NT7- |
|---|---|---|---|---|
| Purified | LP03 | HEK | 14CT20 | 9CT20 |
| FH (10⁻¹² mol) | Specific lysis (%) | | | |
| 0 | 100 | 106.3 | 73.2 | 58.3 |
| 14.58 | 101.4 | 35 | 3.2 | 3.2 |
| 21.87 | 29.5 | 0 | 4 | 0 |
| 29.16 | 8.4 | 0 | 4.7 | 0 |
| 44.74 | 2.7 | 0 | 2.3 | 17.9 |

| | Control | | pCEP4-1NT7- | pCEP4-1NT8- |
|---|---|---|---|---|
| Purified | LP03 | HEK | 11CT20 | 10CT20 |
| FH (10⁻¹² mol) | Specific lysis (%) | | | |
| 0 | 100 | 103.7 | 94.8 | 96.9 |
| 14.58 | | 66.8 | 11.1 | 0 |
| 21.87 | | 7.7 | 2.1 | 1.5 |
| 29.16 | | 3.1 | 0 | 0 |
| 44.74 | 2.5 | 3.7 | 1.1 | 0 |

| | Control | | pCEP4-1NT8- | pCEP4-1NT7- |
|---|---|---|---|---|
| Purified | LP03 | HEK | 11CT20 | 16CT20 |
| FH (10⁻¹² mol) | Specific lysis (%) | | | |
| 0 | 100 | 110.5 | 89.6 | 94.7 |
| 14.58 | | 63 | 37 | 64.6 |
| 21.87 | | 17.8 | 0.3 | 31.3 |
| 29.16 | | 0 | 0 | 20.9 |
| 44.74 | 8.4 | 0 | 0 | 0 |

| | Control | | pCEP4-1NT4- | pCEP4-1NT4- |
|---|---|---|---|---|
| Purified | LP03 | HEK | 19CT20 | 16CT20 |
| FH (10⁻¹² mol) | Specific lysis (%) | | | |
| 0 | 100 | 105.9 | 100.2 | 116.3 |
| 14.58 | | 37.2 | 2.6 | 0 |
| 21.87 | | 5.7 | 0 | 3.1 |
| 29.16 | | 0 | 0 | 0 |
| 44.74 | 0 | 0.5 | 0 | 0 |

| | Control | | pCEP4-1NT9- |
|---|---|---|---|
| Purified | LP03 | HEK | 16CT20 |
| FH (10⁻¹² mol) | Specific lysis (%) | | |
| 0 | 100 | 117.9 | 94.7 |
| 14.58 | | 25.8 | 42.6 |
| 21.87 | | 0 | 33.4 |
| 29.16 | | 0 | 21.6 |
| 44.74 | 3.5 | 0 | 3 |

The test results are similar for the rFH PER.C6 (14-HFACEX-1446-042) and rFH HEK (12-HFACEX-1446-072) samples and consistent with plasma FH (control LP03): dose-effect with total protection of hemolysis at the highest factor H concentration.

The various factor H fragments according to the invention show to be very active with inhibition of specific lysis superior to whole rFH from the lowest concentration tested. For two fragments, 1NT9-16CT20 and 1NT7-16CT20, one observes in an extremely interesting manner a profile closer to the plasma factor H LP03.

1. A recombinant protein having factor H activity, comprising, from the N-terminus to the C-terminus, a first amino acid sequence comprising at least SCR1 to SCR4 of factor H, and a second amino acid sequence comprising at least SCR19 and SCR20 of factor H, said recombinant protein not being a natural factor H, and provided that if the first sequence consists of SCR1 to SCR4 and the second sequence consists of SCR19 and SCR20, the linker is a synthetic linker.

2. The recombinant protein according to claim 1, the first and/or the second amino acid sequence further comprising one or more other rearranged SCR domains of factor H.

3. The recombinant protein according to claim 1, the first and the second amino acid sequence being linked together by a non-natural linker, in particular a G-A-S-G linker.

4. The recombinant protein according to claim 1, the first or the second amino acid sequence comprising at least SCR12, SCR13 and SCR14 of factor H, or the first and the second amino acid sequence not comprising any of SCR12, SCR13 and SCR14.

5. The recombinant protein according to claim 1, the first sequence comprising SCR1 to SCR4 of factor H, and the second sequence is selected from the group consisting of:
SCR16 to SCR20,
SCR15 to SCR20;
-SCR10 to SCR20; and
SCR8 to SCR20 of factor H.

6. The recombinant protein according to claim 1, the first sequence comprising SCR1 to SCR7 of factor H, and the second sequence comprising SCR16 to SCR20 of factor H, said second sequence being preferably selected from the group consisting of:
SCR15 to SCR20;
SCR14 to SCR20;
SCR11 to SCR20;
SCR10 to SCR20;
SCR9 to SCR20; and
SCR8 to SCR20 of factor H.

7. The recombinant protein according to claim 1, the first sequence comprising SCR1 to SCR8 of factor H, and the second sequence comprising SCR16 to SCR20 of factor H, said second sequence preferably comprising SCR10 to SCR20 of factor H.

8. The recombinant protein according to claim 1, the first sequence comprises SCR1 to SCR4 of factor H and the second sequence comprises SCR16 to SCR20 of factor H, a third sequence comprising SCR7 of factor H being between the first and the second sequence.

9. The recombinant protein according to claim 1, the first amino acid sequence comprising a signal peptide at the N-terminal position, in particular the natural signal peptide of factor H (SEQ ID NO: 24), the signal peptide of a protein different from factor H, or the signal peptide SP-MB7 represented in the sequence SEQ ID NO: 25.

10. The recombinant protein according to claim 1, having the sequence selected from the sequences represented in SEQ ID NO: 26 to 84 and 159.

11. A nucleic acid construct encoding a recombinant protein according to claim 1.

12. The nucleic acid construct according to claim 11, comprising a unique restriction site between the two nucleic acid sequences encoding the first and the second amino acid sequence of the recombinant protein, in particular a NheI site present in the portion encoding a G-A-S-G linker.

13. A vector comprising a nucleic acid construct according to claim 11.

14. A host cell comprising the nucleic acid construct according to claim 11.

15. A nonhuman transgenic animal comprising the nucleic acid construct according to claim 11.

16. A host cell comprising the vector according to claim 13.

17. A nonhuman transgenic animal comprising the vector according to claim 13.


SEQUENCE LISTING (159 sequences)

SEQ ID NO: 1 - protein, 1213 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 1213 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 3 - protein, 4 aa, Artificial Sequence
  linker
GASG

SEQ ID NO: 4 - protein, 60 aa, Homo sapiens
CNELPPRRNTEILTGSWSDQTYPEGTQAIYKCRPGYRSLGNVIMVCRKGEWVALNPLRKC

SEQ ID NO: 5 - protein, 57 aa, Homo sapiens
CGHPGDTPFGTFTLTGGNVFEYGVKAVYTCNEGYQLLGEINYRECDTDGWTNDIPIC

SEQ ID NO: 6 - protein, 60 aa, Homo sapiens
CLPVTAPENGKIVSSAMEPDREYHFGQAVRFVCNSGYKIEGDEEMHCSDDGFWSKEKPKC

SEQ ID NO: 7 - protein, 53 aa, Homo sapiens
CKSPDVINGSPISQKIIYKENERFQYKCNMGYEYSERGDAVCTESGWRPLPSC

SEQ ID NO: 8 - protein, 54 aa, Homo sapiens
CDNPYIPNGDYSPLRIKHRTGDEITYQCRNGFYPATRGNTAKCTSTGWIPAPRC

SEQ ID NO: 9 - protein, 61 aa, Homo sapiens
CDYPDIKHGGLYHENMRRPYFPVAVGKYYSYYCDEHFETPSGSYWDHIHCTQDGWSPAVPC

SEQ ID NO: 10 - protein, 54 aa, Homo sapiens
CYFPYLENGYNQNYGRKFVQGKSIDVACHPGYALPKAQTTVTCMENGWSPTPRC

SEQ ID NO: 11 - protein, 58 aa, Homo sapiens
CSKSSIDIENGFISESQYTYALKEKAKYQCKLGYVTADGETSGSITCGKDGWSAQPTC

SEQ ID NO: 12 - protein, 56 aa, Homo sapiens
CDIPVFMNARTKNDFTWFKLNDTLDYECHDGYESNTGSTTGSIVCGYNGWSDLPIC

SEQ ID NO: 13 - protein, 55 aa, Homo sapiens
CELPKIDVHLVPDRKKDQYKVGEVLKFSCKPGFTIVGPNSVQCYHFGLSPDLPIC

SEQ ID NO: 14 - protein, 55 aa, Homo sapiens
CGPPPELLNGNVKEKTKEEYGHSEVVEYYCNPRFLMKGPNKIQCVDGEWTTLPVC

SEQ ID NO: 15 - protein, 54 aa, Homo sapiens
CGDIPELEHGWAQLSSPPYYYGDSVEFNCSESFTMIGHRSITCIHGVWTQLPQC

SEQ ID NO: 16 - protein, 51 aa, Homo sapiens
CKSSNLIILEEHLKNKKEFDHNSNIRYRCRGKEGWIHTVCINGRWDPEVNC

SEQ ID NO: 17 - protein, 54 aa, Homo sapiens
CPPPPQIPNSHNMTTTLNYRDGEKVSVLCQENYLIQEGEEITCKDGRWQSIPLC

SEQ ID NO: 18 - protein, 57 aa, Homo sapiens
CSQPPQIEHGTINSSRSSQESYAHGTKLSYTCEGGFRISEENETTCYMGKWSSPPQC

SEQ ID NO: 19 - protein, 54 aa, Homo sapiens
CKSPPEISHGVVAHMSDSYQYGEEVTYKCFEGFGIDGPAIAKCLGEKWSHPPSC

SEQ ID NO: 20 - protein, 55 aa, Homo sapiens
CLSLPSFENAIPMGEKKDVYKAGEQVTYTCATYYKMDGASNVTCINSRWTGRPTC

SEQ ID NO: 21 - protein, 55 aa, Homo sapiens
CVNPPTVQNAYIVSRQMSKYPSGERVRYQCRSPYEMFGDEEVMCLNGNWTEPPQC

SEQ ID NO: 22 - protein, 55 aa, Homo sapiens
CGPPPPIDNGDITSFPLSVYAPASSVEYQCQNLYQLEGNKRITCRNGQWSEPPKC

SEQ ID NO: 23 - protein, 65 aa, Homo sapiens
CVISREIMENYNIALRWTAKQKLYSRTGESVEFVCKRGYRLSSRSHTLRTTCWDGKLEYPTCAKR

SEQ ID NO: 24 - protein, 18 aa, Homo sapiens
MRLLAKIICLMLWAICVA

SEQ ID NO: 25 - protein, 18 aa, Artificial Sequence
  peptide signal
MRWSWIFLLLLSITSANA

SEQ ID NO: 26 - protein, 402 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHH

SEQ ID NO: 27 - protein, 581 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 28 - protein, 642 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 29 - protein, 701 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 30 - protein, 759 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 31 - protein, 821 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 32 - protein, 882 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 33 - protein, 943 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGCELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 34 - protein, 1003 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 35 - protein, 1064 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 36 - protein, 584 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 37 - protein, 762 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 38 - protein, 823 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 39 - protein, 882 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 40 - protein, 940 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 41 - protein, 1002 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 42 - protein, 1063 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 43 - protein, 1124 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGCELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 44 - protein, 1184 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 45 - protein, 1245 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTGASGCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 46 - protein, 645 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSGASGCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 47 - protein, 823 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSGASGCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 48 - protein, 884 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSGASGCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 49 - protein, 943 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSGASGCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 50 - protein, 1001 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSGASGCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 51 - protein, 1063 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSGASGCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 52 - protein, 1124 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSGASGCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 53 - protein, 1185 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSGASGCELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 54 - protein, 1245 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSGASGCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 55 - protein, 705 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYEREGASGCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 56 - protein, 883 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYEREGASGCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 57 - protein, 944 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYEREGASGCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 58 - protein, 1003 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYEREGASGCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 59 - protein, 1061 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYEREGASGCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 60 - protein, 1123 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYEREGASGCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 61 - protein, 1184 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYEREGASGCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 62 - protein, 1245 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYEREGASGCELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 63 - protein, 766 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSGASGCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 64 - protein, 944 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSGASGCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 65 - protein, 1005 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSGASGCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 66 - protein, 1064 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSGASGCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 67 - protein, 1122 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSGASGCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 68 - protein, 1184 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSGASGCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 69 - protein, 1245 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSGASGCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 70 - protein, 827 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTGASGCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 71 - protein, 1005 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTGASGCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 72 - protein, 1066 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTGASGCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 73 - protein, 1125 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTGASGCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 74 - protein, 1183 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTGASGCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 75 - protein, 1245 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTGASGCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 76 - protein, 889 aa, Artificial Sequence
  fragment FH
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKGASGCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 77 - protein, 1067 aa, Artificial Sequence
  fragment FH
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKGASGCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 78 - protein, 1128 aa, Artificial Sequence
  fragment FH
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKGASGCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 79 - protein, 1187 aa, Artificial Sequence
  fragment FH
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKGASGCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 80 - protein, 1245 aa, Artificial Sequence
  fragment FH
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKGASGCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 81 - protein, 947 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLGASGCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 82 - protein, 1125 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLGASGCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 83 - protein, 1186 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLGASGCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 84 - protein, 1245 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLGASGCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 85 - DNA, 3699 bp, Artificial Sequence
  source 1..3699: /mol_type=“unassigned DNA” /note=“FH optimized sequence” /organism=“Artificial Sequence”
atgagactgctggctaagattatttgcctgatgctgtgggctatttgcgtcgccgaagactgtaacgaactgcctccaagaagaaacaccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgaggactacctgttgggacggaaaactggaataccccacttgtgctaaacgctgataa

SEQ ID NO: 86 - DNA, 12 bp, Artificial Sequence
  source 1..12: /mol_type=“unassigned DNA” /note=“linker” /organism=“Artificial Sequence”
gccgctagcgcc

SEQ ID NO: 87 - DNA, 54 bp, Homo sapiens
  source 1..54: /mol_type=“unassigned DNA” /organism=“Homo sapiens”
atgagacttctagcaaagattatttgccttatgttatgggctatttgtgtagca

SEQ ID NO: 88 - DNA, 54 bp, Artificial Sequence
  source 1..54: /mol_type=“unassigned DNA” /note=“signal peptide sequence” /organism=“Artificial Sequence”
atgagactgctggctaagattatttgcctgatgctgtgggctatttgcgtcgcc

SEQ ID NO: 89 - DNA, 54 bp, Artificial Sequence
  source 1..54: /mol_type=“unassigned DNA” /note=“signal peptide sequence” /organism=“Artificial Sequence”
atgcgatggtcttggatttttctgctgctgctgtctatcacttctgctaacgct

SEQ ID NO: 90 - DNA, 3699 bp, Artificial Sequence
  source 1..3699: /mol_type=“unassigned DNA” /note=“MD3” /organism=“Artificial Sequence”
atgcgatggtcttggatttttctgctgctgctgtctatcacttctgctaacgctgaagactgtaacgaactgcctccaagaagaaacaccgaaatcctgacagggagttggtcagaccagacctaccccgagggaacacaggcaatctataaatgcagacctgggtacaggagcctgggaaatgtgattatggtctgtaggaagggagagtgggtggccctgaacccactgcggaaatgccagaagagaccatgtggacaccccggcgacacacctttcgggacttttaccctgacaggcgggaacgtgttcgaatatggagtgaaggccgtctacacttgcaatgagggctatcagctgctgggggaaatcaactaccgcgagtgtgacactgatggctggaccaatgatatccctatttgcgaagtggtcaaatgtctgccagtgaccgcacccgagaacgggaagatcgtcagctccgccatggaaccagaccgagagtaccacttcggacaggctgtgcggtttgtctgcaattccggatataagatcgagggcgatgaggaaatgcattgttctgacgatggattctggagtaaggaaaaacctaagtgcgtggagattagctgtaaatcccctgacgtgatcaacggctctccaattagtcagaaaatcatctacaaggagaatgaacgctttcagtataagtgcaacatgggctatgaatactctgagcgaggggatgccgtgtgcaccgagagtggatggcgacccctgccttcctgcgaggaaaaatcttgtgacaacccctacatccctaatggggattattccccactgcgcatcaagcaccgaactggagacgagattacctaccagtgcaggaatggcttctaccccgctacacgcggcaacactgcaaagtgtacttctaccggatggattcccgctccccggtgcacactgaaaccttgtgactacccagatattaagcacggaggcctgtaccatgagaatatgcggagaccatatttccccgtggcagtcggcaagtactattcctactattgcgatgaacactttgagacccccagcggctcctattgggaccacatccattgcacacaggatgggtggtctcctgccgtgccatgcctgcgaaaatgttatttcccttacctggagaacggctacaaccagaattacgggcgcaaatttgtgcagggcaagagcatcgacgtggcttgccatcccggctatgctctgcctaaggcacagaccacagtgacctgtatggagaatgggtggtcccctacaccaaggtgcatccgcgtgaaaacttgttctaagagcagcatcgacattgaaaacgggttcatttctgagagtcagtatacatacgccctgaaagagaaggctaaataccagtgcaagctgggatatgtgacagccgacggcgaaacttcagggagcatcacctgcgggaaagatggatggtccgcccagcctacctgcatcaagtcttgtgacattccagtgtttatgaacgctagaacaaagaacgatttcacttggttcaagctgaatgacactctggattacgaatgccacgacggctatgagtccaataccggatctactaccggcagtatcgtgtgcggctacaacgggtggagcgacctgcccatctgctatgagcgggaatgtgagctgccaaaaattgatgtgcacctggtccccgacagaaagaaagatcagtacaaagtgggcgaggtcctgaaattcagctgcaagcccgggtttaccatcgtgggacctaacagcgtccagtgttatcatttcgggctgtcccccgatctgcctatttgcaaggagcaggtgcagagctgtggaccacctccagaactgctgaacggaaatgtgaaggagaaaacaaaggaggaatacggccacagcgaagtggtcgagtactattgcaatccacggtttctgatgaaaggccccaacaagatccagtgcgtggacggggagtggacaactctgcccgtgtgcatcgtcgaggaatccacctgtggcgatattccagaactggagcatgggtgggcacagctgtcaagccccccttactattacggcgacagcgtggaattcaactgctccgagtcttttacaatgatcggacacagatccatcacttgtattcatggcgtgtggacccagctgccacagtgcgtcgccattgacaaactgaagaaatgcaagtcctctaatctgatcattctggaggaacacctgaagaacaagaaggaattcgatcataactctaatatcaggtaccgctgccgaggaaaggagggctggattcacaccgtgtgcattaatgggaggtgggaccccgaagtcaactgctctatggcacagatccagctgtgcccaccacctccacagattcctaacagtcataatatgaccacaactctgaattacagggacggcgagaaggtgtctgtcctgtgccaggaaaactatctgatccaggagggagaggaaattacctgtaaagatggccgctggcagtcaatccctctgtgcgtggaaaagattccatgtagccagccccctcagatcgagcacggaacaattaatagttcacggagctcccaggagagttacgctcatggcaccaagctgtcatatacatgcgaagggggattcagaatcagcgaggaaaacgagaccacatgttacatgggcaaatggtctagtccaccccagtgcgaagggctgccttgtaagagtcctccagagatttcacacggagtggtcgcacatatgagtgactcatatcagtacggcgaggaagtgacatacaaatgcttcgagggctttgggatcgacggccctgccattgctaaatgtctgggagaaaagtggtcacacccccctagctgcatcaagactgactgtctgtcactgcccagctttgaaaatgctattcctatgggggagaagaaagacgtgtacaaagcaggagagcaggtcacatatacttgcgccacatattacaagatggacggcgcttcaaacgtgacttgtatcaatagccggtggaccgggagacctacatgcagggatactagctgcgtgaacccacccaccgtccagaatgcctacatcgtgagccggcagatgtctaagtatccaagtggcgaacgggtgagataccagtgccgcagcccctatgagatgttcggcgacgaggaagtgatgtgcctgaacgggaattggaccgagcctccacagtgcaaagatagcaccgggaagtgtggaccccctccacccatcgacaacggcgatattacttcatttccactgagcgtgtacgcacccgcctcaagcgtcgaataccagtgccagaacctgtatcagctggagggaaacaagcgcatcacctgtcgaaacggccagtggagtgagcctccaaagtgcctgcacccatgcgtgatctcacgggaaattatggagaactacaatattgcactgagatggactgccaaacagaagctgtatagcaggaccggagaatccgtggagttcgtctgcaagcgaggctaccggctgtcctctagaagccataccctgaggactacctgttgggacggaaaactggaataccccacttgtgctaaacgctgataa

SEQ ID NO: 91 - DNA, 21 bp, Artificial Sequence
  Synthetic Polynucleotide
ctctagcggccgcgcgccacc

SEQ ID NO: 92 - DNA, 74 bp, Artificial Sequence
  Synthetic Polynucleotide
ctctaggatccttatcaatggtggtgatggtggtggctgccgctagcgccagatttttcctcgcaggaaggcag

SEQ ID NO: 93 - DNA, 73 bp, Artificial Sequence
  Synthetic Polynucleotide
ctctaggatccttatcaatggtggtgatggtggtggctgccgctagcgccagttttcacgcggatgcaccttg

SEQ ID NO: 94 - DNA, 72 bp, Artificial Sequence
  Synthetic Polynucleotide
ctctaggatccttatcaatggtggtgatggtggtggctgccgctagcgccagacttgatgcaggtaggctgg

SEQ ID NO: 95 - DNA, 74 bp, Artificial Sequence
  Synthetic Polynucleotide
ctctaggatccttatcaatggtggtgatggtggtggctgccgctagcgccttcccgctcatagcagatgggcag

SEQ ID NO: 96 - DNA, 76 bp, Artificial Sequence
  Synthetic Polynucleotide
ctctaggatccttatcaatggtggtgatggtggtggctgccgctagcgccgctctgcacctgctccttgcaaatag

SEQ ID NO: 97 - DNA, 72 bp, Artificial Sequence
  Synthetic Polynucleotide
ctctaggatccttatcaatggtggtgatggtggtggctgccgctagcgccggtggattcctcgacgatgcac

SEQ ID NO: 98 - DNA, 74 bp, Artificial Sequence
  Synthetic Polynucleotide
ctctaggatccttatcaatggtggtgatggtggtggctgccgctagcgcctttcttcagtttgtcaatggcgac

SEQ ID NO: 99 - DNA, 75 bp, Artificial Sequence
  Synthetic Polynucleotide
ctctaggatccttatcaatggtggtgatggtggtggctgccgctagcgcccagctggatctgtgccatagagcag

SEQ ID NO: 100 - DNA, 47 bp, Artificial Sequence
  Synthetic Polynucleotide
ctctaggatccttatcaatggtggtgatggtggtggctgccgctagc

SEQ ID NO: 101 - DNA, 42 bp, Artificial Sequence
  Synthetic Polynucleotide
ctgccttcctgcgaggaaaaatctggcgctagcggcagccac

SEQ ID NO: 102 - DNA, 41 bp, Artificial Sequence
  Synthetic Polynucleotide
caaggtgcatccgcgtgaaaactggcgctagcggcagccac

SEQ ID NO: 103 - DNA, 40 bp, Artificial Sequence
  Synthetic Polynucleotide
ccagcctacctgcatcaagtctggcgctagcggcagccac

SEQ ID NO: 104 - DNA, 42 bp, Artificial Sequence
  Synthetic Polynucleotide
ctgcccatctgctatgagcgggaaggcgctagcggcagccac

SEQ ID NO: 105 - DNA, 43 bp, Artificial Sequence
  Synthetic Polynucleotide
tatttgcaaggagcaggtgcagagcggcgctagcggcagccac

SEQ ID NO: 106 - DNA, 40 bp, Artificial Sequence
  Synthetic Polynucleotide
gtgcatcgtcgaggaatccaccggcgctagcggcagccac

SEQ ID NO: 107 - DNA, 42 bp, Artificial Sequence
  Synthetic Polynucleotide
gtcgccattgacaaactgaagaaaggcgctagcggcagccac

SEQ ID NO: 108 - DNA, 43 bp, Artificial Sequence
  Synthetic Polynucleotide
ctgctctatggcacagatccagctgggcgctagcggcagccac

SEQ ID NO: 109 - DNA, 23 bp, Artificial Sequence
  Synthetic Polynucleotide
ccattgacgtcaatgggagtttg

SEQ ID NO: 110 - DNA, 20 bp, Artificial Sequence
  Synthetic Polynucleotide
tgtcacactcgcggtagttg

SEQ ID NO: 111 - DNA, 22 bp, Artificial Sequence
  Synthetic Polynucleotide
ttcactgcattctagttgtggt

SEQ ID NO: 112 - DNA, 105 bp, Artificial Sequence
  Synthetic Polynucleotide
ctctagcggccgcgcgccaccatgcgatggtcttggatttttctgctgctgctgtctatcacttctgctaacgctgctagcggctgtggaccccctccacccatc

SEQ ID NO: 113 - DNA, 110 bp, Artificial Sequence
  Synthetic Polynucleotide
ctctagcggccgcgcgccaccatgcgatggtcttggatttttctgctgctgctgtctatcacttctgctaacgctgctagcggctgtaagagtcctccagagatttcaca

SEQ ID NO: 114 - DNA, 105 bp, Artificial Sequence
  Synthetic Polynucleotide
ctctagcggccgcgcgccaccatgcgatggtcttggatttttctgctgctgctgtctatcacttctgctaacgctgctagcggctgtagccagccccctcagatc

SEQ ID NO: 115 - DNA, 106 bp, Artificial Sequence
  Synthetic Polynucleotide
ctctagcggccgcgcgccaccatgcgatggtcttggatttttctgctgctgctgtctatcacttctgctaacgctgctagcggctgcccaccacctccacagattc

SEQ ID NO: 116 - DNA, 109 bp, Artificial Sequence
  Synthetic Polynucleotide
ctctagcggccgcgcgccaccatgcgatggtcttggatttttctgctgctgctgtctatcacttctgctaacgctgctagcggctgcaagtcctctaatctgatcattc

SEQ ID NO: 117 - DNA, 106 bp, Artificial Sequence
  Synthetic Polynucleotide
ctctagcggccgcgcgccaccatgcgatggtcttggatttttctgctgctgctgtctatcacttctgctaacgctgctagcggctgtggcgatattccagaactgg

SEQ ID NO: 118 - DNA, 106 bp, Artificial Sequence
  Synthetic Polynucleotide
ctctagcggccgcgcgccaccatgcgatggtcttggatttttctgctgctgctgtctatcacttctgctaacgctgctagcggctgtggaccacctccagaactgc

SEQ ID NO: 119 - DNA, 106 bp, Artificial Sequence
  Synthetic Polynucleotide
ctctagcggccgcgcgccaccatgcgatggtcttggatttttctgctgctgctgtctatcacttctgctaacgctgctagcggctgtgagctgccaaaaattgatg

SEQ ID NO: 120 - DNA, 109 bp, Artificial Sequence
  Synthetic Polynucleotide
ctctagcggccgcgcgccaccatgcgatggtcttggatttttctgctgctgctgtctatcacttctgctaacgctgctagcggctgtgacattccagtgtttatgaacg

SEQ ID NO: 121 - DNA, 109 bp, Artificial Sequence
  Synthetic Polynucleotide
ctctagcggccgcgcgccaccatgcgatggtcttggatttttctgctgctgctgtctatcacttctgctaacgctgctagcggctgttctaagagcagcatcgacattg

SEQ ID NO: 122 - DNA, 74 bp, Artificial Sequence
  Synthetic Polynucleotide
gagtaggatccttatcaatggtggtgatggtggtggccgcttccgcctctcttagcacaagtagggtattccag

SEQ ID NO: 123 - DNA, 45 bp, Artificial Sequence
  Synthetic Polynucleotide
gagtaggatccttatcaatggtggtgatggtggtggccgcttccg

SEQ ID NO: 124 - DNA, 45 bp, Artificial Sequence
  Synthetic Polynucleotide
ctggaataccctacttgtgctaagagaggcggaagcggccaccac

SEQ ID NO: 125 - DNA, 35 bp, Artificial Sequence
  Synthetic Polynucleotide
ctctagcggccgcgcgccaccatgcgatggtcttg

SEQ ID NO: 126 - DNA, 53 bp, Artificial Sequence
  Synthetic Polynucleotide
cgttagcagaagtgatagacagcagcagcagaaaaatccaagaccatcgcatg

SEQ ID NO: 127 - DNA, 73 bp, Artificial Sequence
  Synthetic Polynucleotide
ctctagcggccgcgcgccaccatgcgatggtcttggatttttctgctgctgctgtctatcacttctgctaacg

SEQ ID NO: 128 - DNA, 47 bp, Artificial Sequence
  Synthetic Polynucleotide
gatgggtggagggggtccacagccgctagcagcgttagcagaagtga

SEQ ID NO: 129 - DNA, 52 bp, Artificial Sequence
  Synthetic Polynucleotide
tgtgaaatctctggaggactcttacagccgctagcagcgttagcagaagtga

SEQ ID NO: 130 - DNA, 47 bp, Artificial Sequence
  Synthetic Polynucleotide
gatctgagggggctggctacagccgctagcagcgttagcagaagtga

SEQ ID NO: 131 - DNA, 48 bp, Artificial Sequence
  Synthetic Polynucleotide
gaatctgtggaggtggtgggcagccgctagcagcgttagcagaagtga

SEQ ID NO: 132 - DNA, 51 bp, Artificial Sequence
  Synthetic Polynucleotide
gaatgatcagattagaggacttgcagccgctagcagcgttagcagaagtga

SEQ ID NO: 133 - DNA, 48 bp, Artificial Sequence
  Synthetic Polynucleotide
ccagttctggaatatcgccacagccgctagcagcgttagcagaagtga

SEQ ID NO: 134 - DNA, 48 bp, Artificial Sequence
  Synthetic Polynucleotide
gcagttctggaggtggtccacagccgctagcagcgttagcagaagtga

SEQ ID NO: 135 - DNA, 48 bp, Artificial Sequence
  Synthetic Polynucleotide
catcaatttttggcagctcacagccgctagcagcgttagcagaagtga

SEQ ID NO: 136 - DNA, 51 bp, Artificial Sequence
  Synthetic Polynucleotide
cgttcataaacactggaatgtcacagccgctagcagcgttagcagaagtga

SEQ ID NO: 137 - DNA, 51 bp, Artificial Sequence
  Synthetic Polynucleotide
caatgtcgatgctgctcttagaacagccgctagcagcgttagcagaagtga

SEQ ID NO: 138 - DNA, 40 bp, Artificial Sequence
  Synthetic Polynucleotide
tggtgatgctcagcagcagcaggaagatccagctccatcg

SEQ ID NO: 139 - DNA, 24 bp, Artificial Sequence
  Synthetic Polynucleotide
ggattcacaccgtgtgcattaatg

SEQ ID NO: 140 - DNA, 21 bp, Artificial Sequence
  Synthetic Polynucleotide
agggaaacaagcgcatcacct

SEQ ID NO: 141 - DNA, 20 bp, Artificial Sequence
  Synthetic Polynucleotide
cagatggctggcaactagaa

SEQ ID NO: 142 - protein, 600 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGSSGKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLGSSGKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH

SEQ ID NO: 143 - protein, 4 aa, Homo sapiens
QKRP

SEQ ID NO: 144 - protein, 4 aa, Homo sapiens
EVVK

SEQ ID NO: 145 - protein, 4 aa, Homo sapiens
VEIS

SEQ ID NO: 146 - protein, 4 aa, Homo sapiens
EEKS

SEQ ID NO: 147 - protein, 4 aa, Homo sapiens
TLKP

SEQ ID NO: 148 - protein, 5 aa, Homo sapiens
IRVKT

SEQ ID NO: 149 - protein, 4 aa, Homo sapiens
YERE

SEQ ID NO: 150 - protein, 6 aa, Homo sapiens
KEQVQS

SEQ ID NO: 151 - protein, 6 aa, Homo sapiens
IVEEST

SEQ ID NO: 152 - protein, 8 aa, Homo sapiens
VAIDKLKK

SEQ ID NO: 153 - protein, 7 aa, Homo sapiens
SMAQIQL

SEQ ID NO: 154 - protein, 5 aa, Homo sapiens
VEKIP

SEQ ID NO: 155 - protein, 4 aa, Homo sapiens
EGLP

SEQ ID NO: 156 - protein, 4 aa, Homo sapiens
IKTD

SEQ ID NO: 157 - protein, 4 aa, Homo sapiens
RDTS

SEQ ID NO: 158 - protein, 6 aa, Homo sapiens
KDSTGK

SEQ ID NO: 159 - protein, 647 aa, Artificial Sequence
  fragment FH
MRWSWIFLLLLSITSANAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGASGLRKCYFPYLENGYNQNYGRKFVQGKSIDVACHPGYALPKAQTTVTCMENGWSPTPRCIRVKTGASGCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGSGHHHHHH